(19) United States
(12) Patent Application Publication
(10) Pub. No.: US 2025/0360064 A1
(43) Pub. Date: Nov. 27, 2025

(54) COMPOSITIONS AND METHODS FOR MOISTURIZING A SKIN REGION

(71) Applicant: JAMRM, LLC, Irving, TX (US)

(72) Inventors: Alisar ZAHR, Irving, TX (US); Tatiana KELLY, Irving, TX (US)

(21) Appl. No.: 19/287,188

(22) Filed: Jul. 31, 2025

Related U.S. Application Data
(63) Continuation of application No. PCT/US2024/014307, filed on Feb. 2, 2024.
(60) Provisional application No. 63/483,202, filed on Feb. 3, 2023.

(51) Int. Cl. A61K 8/06; A61Q 19/00

(52) U.S. Cl. CPC A61K 8/064; A61Q 19/007; A61K 2800/522

(57) ABSTRACT

Provided herein are compositions and methods for increasing moisture in a skin region comprising an internal phase in an external phase, wherein the internal phase, external phase, or both comprise a prebiotic, an antioxidant mixture, and a moisturizer mixture, and wherein the internal phase and the external phase are a reverse emulsion.

### CROSS-REFERENCE TO RELATED APPLICATION

This application is a continuation of International Application No. PCT/US2024/014307 filed Feb. 2, 2024, which claims the benefit of U.S. Provisional Application No. 63/483,202 filed on Feb. 3, 2023, which are hereby incorporated by reference in their entirety.

### BACKGROUND

In the past, specific topical products directed toward genital care and health have been deficient and have existed primarily as medical interventions. Additionally, there is a lack of general awareness about and availability of genital products resulting in inadequate care and undesired symptoms. Current products on the market are insufficient and possess a plethora of shortcomings to be addressed.

### SUMMARY

The compositions and methods described herein comprise improved methods of maintaining or improving the health of a skin region. More specifically, the compositions and methods described herein comprise administering a composition formulated to moisturize a skin region and/or improve symptoms of the skin region.

An aspect of the disclosure described herein comprises a composition comprising: an internal phase in an external phase, wherein the internal phase, the external phase, or both comprise: a prebiotic, an antioxidant mixture, and a moisturizer mixture, and wherein the internal phase and the external phase are a reverse emulsion. In some embodiments, the composition is formulated for external administration to a skin region of a mammal. In some embodiments, the prebiotic comprises xylitol, lactitol, a fructan, a polysaccharide, an oligosaccharide, levan, polymnia sonchifolia root juice, or any combination of two or more thereof. In some embodiments, the fructan, the polysaccharide, and/or oligosaccharide comprise pectin, beta-glucan, inulin, bombax costatum flower extract, or any combination of two or more thereof. In some embodiments, the antioxidant mixture comprises tetrahexyldecyl ascorbate, ubiquinone, resveratrol, epigallocatechin gallate, ellagic acid, ursolic acid, oleanolic acid, chlorogenic acid, rutin, hesperidin, quercetin, bakuchiol, botanical extracts of the leaves, flowers, seeds, fruits, stems, barks or roots of a plant, vitamins or derivatives thereof, or any combination of two or more thereof. In some embodiments, the botanical extracts of any part of the plant including the leaves, flowers, seeds, fruits, stems, barks or roots comprise Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract, or any combination of two or more thereof, and the vitamins or derivatives thereof comprise tocopherol, tocopheryl acetate, ubiquinone, ascorbic acid, magnesium ascorbyl phosphate, tetrahexyldecyl ascorbate, retinol, retinaldehyde, retinyl palmitate, or any combination of two or more thereof. In some embodiments, the antioxidant mixture comprises tocopherol. In some embodiments, the moisturizer mixture comprises dimethicone, shea butter, jojoba esters, Helianthus annuus (sunflower) seed wax; Helianthus annuus (sunflower) seed oil, Simmondsia chinensis (jojoba) seed oil, Olea europaea (olive) oil, Glycine soja (soybean) oil, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, cetyl esters, or any combination of two or more thereof. In some embodiments, the internal phase, external phase, or both comprises dipalmitoyl hydroxyproline. In some embodiments, the internal phase, external phase, or both further comprise a carbohydrate or derivative thereof. In some embodiments, the carbohydrate comprises biosaccharide gum-1. In some embodiments, the internal phase, external phase, or both further comprise one or more excipients. In some embodiments, the one or more excipients comprises caprylic/capric triglyceride, glycerin, squalane, dimethicone, glyceryl behenate/eicosadioate, magnesium sulfate, steareth-2, oleth-2, oleth-7, ceteareth-20, sorbitan oleate, sorbitan stearate, sorbitan isostearate, sorbitan laurate, sorbitan olivate, glyceryl oleate, glyceryl stearate, glyceryl ricinoleate, glyceryl caprylate, glyceryl undecylenate, sucrose distearate, peg-20 methyl glucose sesquistearate, peg-30 polyhydroxystearate, polyglyceryl-2 sesquiisostearate, polyglyceryl-2 sesquioleate, palmitic acid, polyglyceryl-2 sesquicaprylate, polyglyceryl-2 diisostearate, polyglyceryl-2 dipolyhydroxystearate, polyglyceryl-2 oleate, polyglyceryl-2 stearate, polyglyceryl-3 oleate, polyglyceryl-3 behenate, polyglyceryl-3 diisostearate, polyglyceryl-3 triolivate, polyglyceryl-3 ricinolate, diisostearoyl polyglyceryl-3 dimer dilinolate, polyglyceryl-3 polyricinoleate, polyglyceryl-3 dimer dilinoleate, polyglyceryl-3 sorbityl linseedate, polyglyceryl-3 polydimethylsiloxyethyl dimethicone, polyglyceryl-3 methylglucose dioleate, polyglyceryl-4 stearate, polyglyceryl-4 oleate, polyglyceryl-4 isostearate, polyglyceryl-4 diisostearate/polyhydroxy stearate/sebacate, polyglyceryl-4 olivate/polyricinolate, polyglyceryl-6 oleate, polyglyceryl-6 pentaoleate, olive oil polyglyceryl-6 esters, polyglyceryl-6 polyricinolate, polyglyceryl-6 polyhydroxystearate, polyglyceryl-10 hexaoleate, polyglyceryl-10 diolate, polyglyceryl-10 stearate, methyl glucose sesquistearate, methyl glucose dioleate hydrogenated lecithin, cetyl peg/ppg-10/1 dimethicone, peg-10 dimethicone, peg/ppg-20/15 dimethicone, peg/ppg-18/18 dimethicone, ppg-8 dimethicone, peg-9 methyl ether dimethicone, bis-peg/ppg-14/14 dimethicone, dimethicone/peg-10/15 crosspolymer, lauryl peg-9 polydimethylsiloxethyl dimethicone, lauryl dimethicone/polyglycerin-3 crosspolymer, lauryl peg-8 dimethicone, lauryl polyglyceryl-3 polydimethylsiloxethyl dimethicone, cetyl diglyceryl tris(trimethylsiloxy)silyethyl dimethicone, sodium hydroxide, ammonium hydroxide, triethanolamine, aminomethyl propanol, citric acid, phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, tetrasodium glutamate diacetate, disodium EDTA, tetrasodium edta, etidronic acid, trisodium dicarboxymethyl alaninate, trisodium ethylenediamine disuccinate, sodium phytate, or any combination of two or more thereof. In some embodiments, the skin region is vulvar, penile, or scrotal. In some embodiments, the mammal is a human. In some embodiments, the human is female. In some embodiments, the skin region is vulvar. In some embodiments, the human is male. In some embodiments, the skin region is penile or scrotal. In some embodiments, a level of moisture in the skin region is increased relative to a reference level of moisture of the skin region after externally administering the composition. In some embodiments, a redness of the skin region is improved relative to a reference redness of the skin region after externally administering the composition. In some embodiments, the composition is a formulation. In some embodiments, the formulation is a cream, lotion, serum, or solid-stick. In some embodiments, the composition is formulated for topical administration to the skin region. In some embodiments, the human experiencing menopausal symptoms, perimenopausal symptoms, premenopausal symptoms, or postmenopausal symptoms. In some embodiments, the menopausal symptoms, perimenopausal symptoms, premenopausal symptoms, or postmenopausal symptoms comprise dryness, irritation, itch, or redness of the skin region. In some embodiments, the menopausal symptoms, perimenopausal symptoms, premenopausal symptoms, or postmenopausal symptoms are reduced after administering the composition. In some embodiments, the human is premenopausal. In some embodiments, the composition is formulated to be administered at a dosage of about 0.01 grams to 10 grams. In some embodiments, the prebiotic is present in the composition at about 0.001% to about 5%. In some embodiments, the prebiotic is present in the composition at about 3% to about 5%. In some embodiments, the antioxidant mixture is present in the composition at about 0.001% to about 5%. In some embodiments, the antioxidant mixture is present in the composition at about 3% to about 5%. In some embodiments, the moisturizer mixture is present in the composition at about 0.001% to 80%. In some embodiments, the moisturizer mixture is present in the composition at about 40% to 80%. In some embodiments, the internal phase comprises a hydrator or humectant. In some embodiments, the hydrator or humectant comprises hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10, or any combination of two or more thereof. In some embodiments, the internal phase is a water phase. In some embodiments, the external phase is an oil phase or a silicone phase. In some embodiments, the composition comprises a antioxidant mixture. In some embodiments, the internal phase, external phase, or both comprise the antioxidant mixture. In some embodiments, the antioxidant mixture comprises Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract, or any combination of two or more thereof. In some embodiments, a level of smoothness of the skin region is improved relative to a reference level of smoothness of the skin region after externally administering the composition. In some embodiments, a level of irritation of the skin region is improved relative to a reference level of irritation of the skin region after externally administering the composition. In some embodiments, a texture of the skin region is improved relative to a reference texture of the skin region after externally administering the composition. In some embodiments, the skin region is soothed after externally administering the composition. In some embodiments, the composition is externally administered after the skin region is shaved. In some embodiments, the internal phase comprises the antioxidant mixture. In some embodiments, the external phase comprises the antioxidant mixture. In some embodiments, the internal phase comprises the moisturizer mixture. In some embodiments, the external phase comprises the moisturizer mixture. In some embodiments, the internal phase comprises the carbohydrate. In some embodiments, the external phase comprises the carbohydrate. In some embodiments, the internal phase comprises the antioxidant mixture; and the external phase comprises the moisturizer mixture.

Another aspect of the disclosure described herein comprises a method for increasing moisture in a skin region of a mammal, comprising externally administering a composition formulated as a reverse emulsion to the skin region, the composition comprising: an internal phase in an external phase, wherein the internal phase, external phase, or both comprise: a prebiotic; an antioxidant mixture; and a moisturizer mixture. In some embodiments, the prebiotic comprises xylitol, lactitol, a fructan, a polysaccharide, an oligosaccharide, levan, polymnia sonchifolia root juice, or any combination of two or more thereof. In some embodiments, the fructan, the polysaccharide, and/or oligosaccharide comprise pectin, beta-glucan, inulin, bombax costatum flower extract, or any combination of two or more thereof. In some embodiments, the antioxidant mixture comprises tetrahexyldecyl ascorbate, ubiquinone, resveratrol, epigallocatechin gallate, ellagic acid, ursolic acid, oleanolic acid, chlorogenic acid, rutin, hesperidin, quercetin, bakuchiol, botanical extracts of any part of a plant including the leaves, flowers, seeds, fruits, stems, barks, or roots of the plant, vitamins or derivatives thereof, or any combination of two or more thereof. In some embodiments, botanical extracts of any part of the plant including the leaves, flowers, seeds, fruits, stems, barks or roots comprise Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract, or any combination of two or more thereof, and the vitamins comprise tocopherol, tocoopheryl acetate, ubiquinone, ascorbic acid, magnesium ascorbyl phosphate, tetrahexyldecyl ascorbate, retinol, retinaldehyde, retinyl palmitate, or any combination of two or more thereof. In some embodiments, the antioxidant mixture comprises tocopherol. In some embodiments, the moisturizer mixture comprises dimethicone, shea butter, jojoba esters, Helianthus annuus (sunflower) seed wax; Helianthus annuus (sunflower) seed oil, Simmondsia chinensis (jojoba) seed oil, Olea europaea (olive) oil, Glycine soja (soybean) oil, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, cetyl esters, or any combination of two or more thereof. In some embodiments, the internal phase, external phase, or both further comprise dipalmitoyl hydroxyproline. In some embodiments, the internal phase, external phase, or both comprises a carbohydrate or derivative thereof. In some embodiments, the carbohydrate comprises biosaccharide gum-1. In some embodiments, the internal phase, external phase, or both further comprise one or more excipients. In some embodiments, the one or more excipients comprises caprylic/capric triglyceride, glycerin, squalane, dimethicone, glyceryl behenate/eicosadioate, magnesium sulfate, steareth-2, oleth-2, oleth-7, ceteareth-20, sorbitan oleate, sorbitan stearate, sorbitan isostearate, sorbitan laurate, sorbitan olivate, glyceryl oleate, glyceryl stearate, glyceryl ricinoleate, glyceryl caprylate, glyceryl undecylenate, sucrose distearate, peg-20 methyl glucose sesquistearate, peg-30 polyhydroxystearate, polyglyceryl-2 sesquiisostearate, polyglyceryl-2 sesquioleate, palmitic acid, polyglyceryl-2 sesquicaprylate, polyglyceryl-2 diisostearate, polyglyceryl-2 dipolyhydroxystearate, polyglyceryl-2 oleate, polyglyceryl-2 stearate, polyglyceryl-3 oleate, polyglyceryl-3 behenate, polyglyceryl-3 diisostearate, polyglyceryl-3 triolivate, polyglyceryl-3 ricinolate, diisostearoyl polyglyceryl-3 dimer dilinolate, polyglyceryl-3 polyricinoleate, polyglyceryl-3 dimer dilinoleate, polyglyceryl-3 sorbityl linseedate, polyglyceryl-3 polydimethylsiloxyethyl dimethicone, polyglyceryl-3 methylglucose dioleate, polyglyceryl-4 stearate, polyglyceryl-4 oleate, polyglyceryl-4 isostearate, polyglyceryl-4 diisostearate/polyhydroxy stearate/sebacate, polyglyceryl-4 olivate/polyricinolate, polyglyceryl-6 oleate, polyglyceryl-6 pentaoleate, olive oil polyglyceryl-6 esters, polyglyceryl-6 polyricinolate, polyglyceryl-6 polyhydroxystearate, polyglyceryl-10 hexaoleate, polyglyceryl-10 diolate, polyglyceryl-10 stearate, methyl glucose sesquistearate, methyl glucose dioleate hydrogenated lecithin, cetyl peg/ppg-10/1 dimethicone, peg-10 dimethicone, peg/ppg-20/15 dimethicone, peg/ppg-18/18 dimethicone, ppg-8 dimethicone, peg-9 methyl ether dimethicone, bis-peg/ppg-14/14 dimethicone, dimethicone/peg-10/15 crosspolymer, lauryl peg-9 polydimethylsiloxethyl dimethicone, lauryl dimethicone/polyglycerin-3 crosspolymer, lauryl peg-8 dimethicone, lauryl polyglyceryl-3 polydimethylsiloxethyl dimethicone, cetyl diglyceryl tris(trimethylsiloxy)silyethyl dimethicone, sodium hydroxide, ammonium hydroxide, triethanolamine, aminomethyl propanol, citric acid, phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, tetrasodium glutamate diacetate, disodium EDTA, tetrasodium edta, etidronic acid, trisodium dicarboxymethyl alaninate, trisodium ethylenediamine disuccinate, sodium phytate, or any combination of two or more thereof. In some embodiments, the skin region is vulvar, penile, or scrotal. In some embodiments, the mammal is a human. In some embodiments, the human is female. In some embodiments, the skin region is vulvar. In some embodiments, the human is male. In some embodiments, the skin region is penile or scrotal. In some embodiments, administering the composition to the skin region comprises administering the composition to the skin region at a frequency of once daily, twice daily, thrice daily, four times daily, five times daily, or six times daily. In some embodiments, the frequency is twice daily. In some embodiments, a level of moisture in the skin region is increased relative to a reference level of moisture in the skin region after externally administering the composition. In some embodiments, a redness of the skin region is improved relative to a reference redness of the skin region after externally administering the composition. In some embodiments, the composition is a formulation. In some embodiments, the formulation is a cream, serum, lotion, or solid-stick. In some embodiments, externally administering the composition to the skin region comprises topically applying the composition to the skin region. In some embodiments, the human is experiencing menopausal symptoms, perimenopausal symptoms, premenopausal symptoms, or postmenopausal symptoms. In some embodiments, the menopausal symptoms, perimenopausal symptoms, premenopausal symptoms, or postmenopausal symptoms comprise dryness, irritation, itch, or redness of the skin region. In some embodiments, the menopausal symptoms, perimenopausal symptoms, premenopausal symptoms, or postmenopausal symptoms are reduced after administering the composition. In some embodiments, the human is premenopausal. In some embodiments, externally administering the composition to the skin region comprises administering the composition at a dosage of about 0.1 grams to about 10 grams. In some embodiments, the prebiotic is present in the composition at about 0.001% to about 5%. In some embodiments, the prebiotic is present in the composition at about 3% to about 5%. In some embodiments, the antioxidant mixture is present in the composition at about 0.001% to about 5%. In some embodiments, the antioxidant mixture is present in the composition at about 3% to about 5%. In some embodiments, the moisturizer mixture is present in the composition at about 0.001% to 80%. In some embodiments, the moisturizer mixture is present in the composition at about 40% to 80%. In some embodiments, externally administering the composition results in increased collagen production in the skin region. In some embodiments, the internal phase comprises a hydrator or humectant. In some embodiments, the hydrator or humectant comprises hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10, or any combination of two or more thereof. In some embodiments, the internal phase is a water phase. In some embodiments, the external phase is an oil phase or a silicone phase. In some embodiments, the composition comprises a antioxidant mixture. In some embodiments, the internal phase, external phase, or both comprises the antioxidant mixture. In some embodiments, the antioxidant mixture comprises Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract, or any combination of two or more thereof. In some embodiments, a level of smoothness of the skin region is improved relative to a reference level of smoothness of the skin region after externally administering the composition. In some embodiments, a level of irritation of the skin region is improved relative to a reference level of irritation of the skin region after externally administering the composition. In some embodiments, a texture of the skin region is improved relative to a reference texture of the skin region after externally administering the composition. In some embodiments, the skin region is soothed after externally administering the composition. In some embodiments, the composition is externally administered after the skin region is shaved. In some embodiments, the internal phase comprises the antioxidant mixture. In some embodiments, the external phase comprises the antioxidant mixture. In some embodiments, the internal phase comprises the moisturizer mixture. In some embodiments, the external phase comprises the moisturizer mixture. In some embodiments, the internal phase comprises the carbohydrate. In some embodiments, the external phase comprises the carbohydrate. In some embodiments, the internal phase comprises the antioxidant mixture; and the external phase comprises the moisturizer mixture.

### DETAILED DESCRIPTION

Skin health can often go unrecognized or unmaintained for a variety of reasons. Whether due to lack of viable skin health products, allergies to certain components in skin health products, a disregard for certain aspects of skin health, or lack of knowledge of how to maintain the health of skin, a person's skin or certain skin regions may not receive adequate care. This may be in particular skin regions, such as a person's genitalia, where skin care may not be recognized as necessary or may be seen as taboo.

The present disclosure is directed towards improving or maintaining the health of a person's skin or particular skin regions by improving aspects of the person's skin or skin regions. Certain ingredients, such as antioxidant mixtures, moisturizer mixtures, and prebiotics, help moisturize the skin, promote healthy skin barriers, enhance a balanced skin microbiome, and either keep the skin in a desirable condition or help return the skin to that desirable condition. Even further, formulations of the compositions as reverse emulsions can boost the efficacy of the compositions by increasing its ability to moisturize, maintain healthy skin barriers, enhance a balanced skin microbiome, and even help reduce symptoms of certain conditions, such as menopause.

### Definitions

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

As used herein, “individual” or “subject” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

“Substantially” as the term is used herein means completely or almost completely. For example, a composition that is “substantially free” of a component either has none of the component or contains such a trace amount that any relevant functional property of the composition is unaffected by the presence of the trace amount. For example, a composition that is “substantially pure” has only negligible traces of impurities present.

In various embodiments, a composition as shown in any of the Examples, or among the exemplary compounds, is provided.

Provisos may apply to any of the disclosed categories or embodiments wherein any one or more of the other above disclosed embodiments or species may be excluded from such categories or embodiments.

The terms “a” or “an”, as used in herein means one or more.

Excipients to a substance or excipients of a composition refer to a component that aids the application of a composition and can be included in the compositions of the present disclosure without causing a significant adverse toxicological effect on the patient. Such preparations can be sterilized and, if desired, mixed with auxiliary agents such as emollients, preservatives, stabilizers, emulsifiers, salts, buffers, coloring agent, and/or aromatic substances and the like that do not deleteriously react with the compositions of the disclosure. One of skill in the art will recognize that other pharmaceutical excipients are useful in the present disclosure.

“Individual,” “subject,” or any similar such term refers to a living organism suffering from or prone to a disease or condition. Non-limiting examples include humans, other mammals, bovines, rats, mice, dogs, monkeys, goat, sheep, cows, deer, and other non-mammalian animals. In some embodiments, a subject is human.

Target concentrations will be those concentrations of active compound(s) within compositions or the methods described herein, as measured using the methods described herein or known in the art.

Dosage may vary depending on certain conditions or disorders or symptoms related to a condition or disorder or the health of a skin region. Determination of the proper dosage for a particular situation is within the skill of one skilled in the art.

As used herein, an “excipient” may also refer to a biologically inactive substance that serves various functions in a composition such as a vehicle, medium, stabilizer, preservative, emollients, emulsifier, salt, buffer, coloring agent, and/or aromatic substance and the like that do not deleteriously react with the compositions of the disclosure or other similar inactive ingredient. Non-limiting examples of excipients include pH buffers, emulsifiers, preservatives, antioxidants, diluents, and bulking agents.

### Emulsions and Reverse Emulsions

Emulsions and reverse emulsions are mixtures of two immiscible substances, such as oil and water, that stay in a mixed state. In some cases, the two immiscible substances stay together due an emulsifier, which can be a third substance or an action (e.g., homogenization or colloid milling) that helps the immiscible substances stay mixed. In some cases, the two immiscible substances may be referred to as phases. For example, the first immiscible substance (e.g., water) may be a first phase, and a second immiscible substance (e.g., oil) may be a second phase.

In some cases, one phase may be in another phase, such as a dispersion of oily materials as an internal phase (e.g., a first phase) in an aqueous medium serving as an external phase (e.g., a second phase), or a dispersion of water as an internal phase (e.g., a first phase) in an oil or silicone based material serving as an external phase. As described herein, emulsions are considered to have a first phase (e.g., an oil phase, oil-based phase, silicone-phase, or silicone-based phase) in a water phase, and reverse emulsions are considered to have a water phase or a water-based phase in a first phase (e.g., an oil phase, oil-based phase, silicone-phase, or silicone-based phase). Examples of oil phases, oil-based phases, silicone phases, or silicone-based phases include but are not limited to mineral oils, vegetable oils, esters, and silicone waxes and liquids. As described herein, emulsions are characterized as having a water phase or a water-based phase as an external phase and having a non-water phase (e.g., an oil phase, oil-based phase, siliconephase, or silicone-based phase) as an internal phase, and reverse emulsions are characterized a non-water phase (e.g., an oil phase, oil-based phase, siliconephase, or silicone-based phase) as an external phase.

Reverse emulsions, in particular, have certain advantages that are not captured by emulsions. For example, reverse emulsions may have an improved feel, improved spreadability, improved breathability, improved moisturization, and substantivity relative to emulsions. In some embodiments described herein, the compositions are formulated as reverse emulsions.

### Compositions

Provided herein, in some embodiments, are compositions for maintaining or improving the health of skin region. In some embodiments, the compositions are formulations or include formulations. In some embodiments, the compositions are solid or liquid. In some embodiments, the composition is a reverse emulsion. In some embodiments, the composition is an emulsion. In some embodiments, the composition has an internal phase and an external phase. In some embodiments, the internal phase is in the external phase. In some embodiments, the internal phase is water. In some embodiments, the external phase is oil. In some embodiments, the external phase is silicone or silicone-based. In some embodiments, the internal phase is a water phase and the external phase is an oil phase or an oil-based phase. In some embodiments, the internal phase is a water phase and the external phase is a silicone phase or a silicone-based phase. In some embodiments, the composition can be applied to a skin region. In some embodiments, the skin region can be of a mammal, including but not limited to humans. In some embodiments, the skin region can be related to human genitalia. In some embodiments, the skin region is of the vulva, the penis, or the scrotum.

Provided herein are compositions for external application to a skin region. In some embodiments, the composition is a reverse emulsion. In some embodiments, the reverse emulsion comprises a first phase and a second phase. In some embodiments, the first phase is an internal phase and the second phase is an external phase.

In some embodiments, the internal phase comprises about 0.01% to about 5% w/w of the composition. In some embodiments, the first phase comprises about 0.01% to about 0.05%, about 0.01% to about 0.1%, about 0.01% to about 0.5%, about 0.01% to about 1%, about 0.01% to about 5%, about 0.05% to about 0.1%, about 0.05% to about 0.5%, about 0.05% to about 1%, about 0.05% to about 5%, about 0.1% to about 0.5%, about 0.1% to about 1%, about 0.1% to about 5%, about 0.5% to about 1%, about 0.5% to about 5%, or about 1% to about 5% w/w of the composition. In some embodiments, the first phase comprises about 0.01%, about 0.05%, about 0.1%, about 0.5%, about 1%, or about 5% w/w of the composition. In some embodiments, the first phase comprises at least about 0.01%, about 0.05%, about 0.1%, about 0.5%, or about 1% w/w of the composition. In some embodiments, the first phase comprises at most about 0.05%, about 0.1%, about 0.5%, about 1%, or about 5% w/w of the composition. In some embodiments, the first phase comprises about 5% to about 50% w/w of the composition. In some embodiments, the first phase comprises about 5% to about 7.5%, about 5% to about 10%, about 5% to about 12.5%, about 5% to about 15%, about 5% to about 20%, about 5% to about 25%, about 5% to about 30%, about 5% to about 35%, about 5% to about 40%, about 5% to about 45%, about 5% to about 50%, about 7.5% to about 10%, about 7.5% to about 12.5%, about 7.5% to about 15%, about 7.5% to about 20%, about 7.5% to about 25%, about 7.5% to about 30%, about 7.5% to about 35%, about 7.5% to about 40%, about 7.5% to about 45%, about 7.5% to about 50%, about 10% to about 12.5%, about 10% to about 15%, about 10% to about 20%, about 10% to about 25%, about 10% to about 30%, about 10% to about 35%, about 10% to about 40%, about 10% to about 45%, about 10% to about 50%, about 12.5% to about 15%, about 12.5% to about 20%, about 12.5% to about 25%, about 12.5% to about 30%, about 12.5% to about 35%, about 12.5% to about 40%, about 12.5% to about 45%, about 12.5% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50% w/w of the composition. In some embodiments, the first phase comprises about 5%, about 7.5%, about 10%, about 12.5%, about 15%, about 20%, about 25%, about 30%, about 35%, about 40%, about 45%, or about 50% w/w of the composition. In some embodiments, the first phase comprises at least about 5%, about 7.5%, about 10%, about 12.5%, about 15%, about 20%, about 25%, about 30%, about 35%, about 40%, or about 45% w/w of the composition. In some embodiments, the first phase comprises at most about 7.5%, about 10%, about 12.5%, about 15%, about 20%, about 25%, about 30%, about 35%, about 40%, about 45%, or about 50% w/w of the composition.

In some embodiments, the internal phase is a water phase. In some embodiments, the water phase is water. In some embodiments, the water phase is water-based.

In some embodiments, the composition includes a prebiotic. In some embodiments, the internal phase includes the prebiotic. In some embodiments, the external phase includes the prebiotic. In some embodiments, both the internal phase and the external phase includes the prebiotic. In some embodiments, the prebiotic is xylitol, lactitol, or both. In some embodiments, the prebiotic is xylitol, lactitol, or any combination thereof. In some embodiments, the prebiotic is one or more of xylitol, lactitol, a fructan, one or more polysaccharides and/or oligosaccharides (e.g., pectin, beta-glucan, inulin, bombax costatum flower extract), levan, polymnia sonchifolia root juice, alpha-glucan oligosaccharide or any combination of two or more thereof. In some embodiments, the prebiotic regulates commensal and pathogenic bacteria of a skin region to which the composition is applied. In some embodiments, the prebiotic promotes the activity of beneficial bacteria of the skin region. In some embodiments, the prebiotic is non-fermentable.

In some embodiments, the prebiotic comprises about 0.001% to about 0.01% w/w of the composition. In some embodiments, the prebiotic comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the prebiotic comprises about 0.001%, about 0.002%, about 0.003%, about 0.004%, about 0.005%, about 0.006%, about 0.007%, about 0.008%, about 0.009%, or about 0.01% w/w of the composition. In some embodiments, the prebiotic comprises at least about 0.001%, about 0.002%, about 0.003%, about 0.004%, about 0.005%, about 0.006%, about 0.007%, about 0.008%, or about 0.009% w/w of the composition. In some embodiments, the prebiotic comprises at most about 0.002%, about 0.003%, about 0.004%, about 0.005%, about 0.006%, about 0.007%, about 0.008%, about 0.009%, or about 0.01% w/w of the composition. In some embodiments, the prebiotic comprises about 0.01% to about 0.1% w/w of the composition. In some embodiments, the prebiotic comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the prebiotic comprises about 0.01%, about 0.02%, about 0.03%, about 0.04%, about 0.05%, about 0.06%, about 0.07%, about 0.08%, about 0.09%, or about 0.1% w/w of the composition. In some embodiments, the prebiotic comprises at least about 0.01%, about 0.02%, about 0.03%, about 0.04%, about 0.05%, about 0.06%, about 0.07%, about 0.08%, or about 0.09% w/w of the composition. In some embodiments, the prebiotic comprises at most about 0.02%, about 0.03%, about 0.04%, about 0.05%, about 0.06%, about 0.07%, about 0.08%, about 0.09%, or about 0.1% w/w of the composition. In some embodiments, the prebiotic comprises about 0.1% to about 1% w/w of the composition. In some embodiments, the prebiotic comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the prebiotic comprises about 0.1%, about 0.2%, about 0.3%, about 0.4%, about 0.5%, about 0.6%, about 0.7%, about 0.8%, about 0.9%, or about 1% w/w of the composition. In some embodiments, the prebiotic comprises at least about 0.1%, about 0.2%, about 0.3%, about 0.4%, about 0.5%, about 0.6%, about 0.7%, about 0.8%, or about 0.9% w/w of the composition. In some embodiments, the prebiotic comprises at most about 0.2%, about 0.3%, about 0.4%, about 0.5%, about 0.6%, about 0.7%, about 0.8%, about 0.9%, or about 1% w/w of the composition. In some embodiments, the prebiotic comprises about 0.1% to about 0.25% w/w of the composition. In some embodiments, the prebiotic comprises about 0.1% to about 0.15%, about 0.1% to about 0.2%, about 0.1% to about 0.25%, about 0.15% to about 0.2%, about 0.15% to about 0.25%, or about 0.2% to about 0.25% w/w of the composition. In some embodiments, the prebiotic comprises about 0.1%, about 0.15%, about 0.2%, or about 0.25% w/w of the composition. In some embodiments, the prebiotic comprises at least about 0.1%, about 0.15%, or about 0.2% w/w of the composition. In some embodiments, the prebiotic comprises at most about 0.15%, about 0.2%, or about 0.25% w/w of the composition. In some embodiments, the prebiotic comprises about 0.25% to about 5% w/w of the composition. In some embodiments, the prebiotic comprises about 0.25% to about 0.5%, about 0.25% to about 1%, about 0.25% to about 1.5%, about 0.25% to about 2%, about 0.25% to about 2.5%, about 0.25% to about 3%, about 0.25% to about 3.5%, about 0.25% to about 4%, about 0.25% to about 4.5%, about 0.25% to about 5%, about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5% w/w of the composition. In some embodiments, the prebiotic comprises about 0.25%, about 0.5%, about 1%, about 1.5%, about 2%, about 2.5%, about 3%, about 3.5%, about 4%, about 4.5%, or about 5% w/w of the composition. In some embodiments, the prebiotic comprises at least about 0.25%, about 0.5%, about 1%, about 1.5%, about 2%, about 2.5%, about 3%, about 3.5%, about 4%, or about 4.5% w/w of the composition. In some embodiments, the prebiotic comprises at most about 0.5%, about 1%, about 1.5%, about 2%, about 2.5%, about 3%, about 3.5%, about 4%, about 4.5%, or about 5% w/w of the composition. In some embodiments, the prebiotic comprises about 5% to about 20% w/w of the composition. In some embodiments, the prebiotic comprises about 5% to about 6%, about 5% to about 7%, about 5% to about 8%, about 5% to about 9%, about 5% to about 10%, about 5% to about 12%, about 5% to about 14%, about 5% to about 16%, about 5% to about 18%, about 5% to about 20%, about 6% to about 7%, about 6% to about 8%, about 6% to about 9%, about 6% to about 10%, about 6% to about 12%, about 6% to about 14%, about 6% to about 16%, about 6% to about 18%, about 6% to about 20%, about 7% to about 8%, about 7% to about 9%, about 7% to about 10%, about 7% to about 12%, about 7% to about 14%, about 7% to about 16%, about 7% to about 18%, about 7% to about 20%, about 8% to about 9%, about 8% to about 10%, about 8% to about 12%, about 8% to about 14%, about 8% to about 16%, about 8% to about 18%, about 8% to about 20%, about 9% to about 10%, about 9% to about 12%, about 9% to about 14%, about 9% to about 16%, about 9% to about 18%, about 9% to about 20%, about 10% to about 12%, about 10% to about 14%, about 10% to about 16%, about 10% to about 18%, about 10% to about 20%, about 12% to about 14%, about 12% to about 16%, about 12% to about 18%, about 12% to about 20%, about 14% to about 16%, about 14% to about 18%, about 14% to about 20%, about 16% to about 18%, about 16% to about 20%, or about 18% to about 20% w/w of the composition. In some embodiments, the prebiotic comprises about 5%, about 6%, about 7%, about 8%, about 9%, about 10%, about 12%, about 14%, about 16%, about 18%, or about 20% w/w of the composition. In some embodiments, the prebiotic comprises at least about 5%, about 6%, about 7%, about 8%, about 9%, about 10%, about 12%, about 14%, about 16%, or about 18% w/w of the composition. In some embodiments, the prebiotic comprises at most about 6%, about 7%, about 8%, about 9%, about 10%, about 12%, about 14%, about 16%, about 18%, or about 20% w/w of the composition. In some embodiments, the prebiotic comprises about 20% to about 40% w/w of the composition. In some embodiments, the prebiotic comprises about 20% to about 24%, about 20% to about 28%, about 20% to about 32%, about 20% to about 36%, about 20% to about 40%, about 24% to about 28%, about 24% to about 32%, about 24% to about 36%, about 24% to about 40%, about 28% to about 32%, about 28% to about 36%, about 28% to about 40%, about 32% to about 36%, about 32% to about 40%, or about 36% to about 40% w/w of the composition. In some embodiments, the prebiotic comprises about 20%, about 24%, about 28%, about 32%, about 36%, or about 40% w/w of the composition. In some embodiments, the prebiotic comprises at least about 20%, about 24%, about 28%, about 32%, or about 36% w/w of the composition. In some embodiments, the prebiotic comprises at most about 24%, about 28%, about 32%, about 36%, or about 40% w/w of the composition.

In some embodiments, the composition further includes an antioxidant mixture. In some embodiments, the internal phase includes the antioxidant mixture. In some embodiments, the external phase includes the antioxidant mixture. In some embodiments, the internal phase and the external phase both include the antioxidant mixture. In some embodiments, the antioxidant mixture is Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract or any combination of two or more thereof. in some embodiments, skin regions to which a composition including an antioxidant mixture is applied experience a strengthened and balanced protective barrier of the skin region relative to a protective barrier of a skin region to which a composition including a antioxidant mixture is not applied.

In some embodiments, the antioxidant mixture comprises about 0.001% to about 8% w/w of the composition. In some embodiments, the antioxidant mixture comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the antioxidant mixture comprises about 0.001%, about 0.002%, about 0.003%, about 0.004%, about 0.005%, about 0.006%, about 0.007%, about 0.008%, about 0.009%, or about 0.01% w/w of the composition. In some embodiments, the antioxidant mixture comprises at least about 0.001%, about 0.002%, about 0.003%, about 0.004%, about 0.005%, about 0.006%, about 0.007%, about 0.008%, or about 0.009% w/w of the composition. In some embodiments, the antioxidant mixture comprises at most about 0.002%, about 0.003%, about 0.004%, about 0.005%, about 0.006%, about 0.007%, about 0.008%, about 0.009%, or about 0.01% w/w of the composition. In some embodiments, the antioxidant mixture comprises about 0.01% to about 0.1% w/w of the composition. In some embodiments, the antioxidant mixture comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the antioxidant mixture comprises about 0.01%, about 0.02%, about 0.03%, about 0.04%, about 0.05%, about 0.06%, about 0.07%, about 0.08%, about 0.09%, or about 0.1% w/w of the composition. In some embodiments, the antioxidant mixture comprises at least about 0.01%, about 0.02%, about 0.03%, about 0.04%, about 0.05%, about 0.06%, about 0.07%, about 0.08%, or about 0.09% w/w of the composition. In some embodiments, the antioxidant mixture comprises at most about 0.02%, about 0.03%, about 0.04%, about 0.05%, about 0.06%, about 0.07%, about 0.08%, about 0.09%, or about 0.1% w/w of the composition. In some embodiments, the antioxidant mixture comprises about 0.1% to about 1% w/w of the composition. In some embodiments, the antioxidant mixture comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the antioxidant mixture comprises about 0.1%, about 0.2%, about 0.3%, about 0.4%, about 0.5%, about 0.6%, about 0.7%, about 0.8%, about 0.9%, or about 1% w/w of the composition. In some embodiments, the antioxidant mixture comprises at least about 0.1%, about 0.2%, about 0.3%, about 0.4%, about 0.5%, about 0.6%, about 0.7%, about 0.8%, or about 0.9% w/w of the composition. In some embodiments, the antioxidant mixture comprises at most about 0.2%, about 0.3%, about 0.4%, about 0.5%, about 0.6%, about 0.7%, about 0.8%, about 0.9%, or about 1% w/w of the composition. In some embodiments, the antioxidant mixture comprises about 0.1% to about 0.25% w/w of the composition. In some embodiments, the antioxidant mixture comprises about 0.1% to about 0.15%, about 0.1% to about 0.2%, about 0.1% to about 0.25%, about 0.15% to about 0.2%, about 0.15% to about 0.25%, or about 0.2% to about 0.25% w/w of the composition. In some embodiments, the antioxidant mixture comprises about 0.1%, about 0.15%, about 0.2%, or about 0.25% w/w of the composition. In some embodiments, the antioxidant mixture comprises at least about 0.1%, about 0.15%, or about 0.2% w/w of the composition. In some embodiments, the antioxidant mixture comprises at most about 0.15%, about 0.2%, or about 0.25% w/w of the composition. In some embodiments, the antioxidant mixture comprises about 0.25% to about 5% w/w of the composition. In some embodiments, the antioxidant mixture comprises about 0.25% to about 0.5%, about 0.25% to about 1%, about 0.25% to about 1.5%, about 0.25% to about 2%, about 0.25% to about 2.5%, about 0.25% to about 3%, about 0.25% to about 3.5%, about 0.25% to about 4%, about 0.25% to about 4.5%, about 0.25% to about 5%, about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5% w/w of the composition. In some embodiments, the antioxidant mixture comprises about 0.25%, about 0.5%, about 1%, about 1.5%, about 2%, about 2.5%, about 3%, about 3.5%, about 4%, about 4.5%, or about 5% w/w of the composition. In some embodiments, the antioxidant mixture comprises at least about 0.25%, about 0.5%, about 1%, about 1.5%, about 2%, about 2.5%, about 3%, about 3.5%, about 4%, or about 4.5% w/w of the composition. In some embodiments, the antioxidant mixture comprises at most about 0.5%, about 1%, about 1.5%, about 2%, about 2.5%, about 3%, about 3.5%, about 4%, about 4.5%, or about 5% w/w of the composition. In some embodiments, the antioxidant mixture comprises about 5% to about 20% w/w of the composition. In some embodiments, the antioxidant mixture comprises about 5% to about 6%, about 5% to about 7%, about 5% to about 8%, about 5% to about 9%, about 5% to about 10%, about 5% to about 12%, about 5% to about 14%, about 5% to about 16%, about 5% to about 18%, about 5% to about 20%, about 6% to about 7%, about 6% to about 8%, about 6% to about 9%, about 6% to about 10%, about 6% to about 12%, about 6% to about 14%, about 6% to about 16%, about 6% to about 18%, about 6% to about 20%, about 7% to about 8%, about 7% to about 9%, about 7% to about 10%, about 7% to about 12%, about 7% to about 14%, about 7% to about 16%, about 7% to about 18%, about 7% to about 20%, about 8% to about 9%, about 8% to about 10%, about 8% to about 12%, about 8% to about 14%, about 8% to about 16%, about 8% to about 18%, about 8% to about 20%, about 9% to about 10%, about 9% to about 12%, about 9% to about 14%, about 9% to about 16%, about 9% to about 18%, about 9% to about 20%, about 10% to about 12%, about 10% to about 14%, about 10% to about 16%, about 10% to about 18%, about 10% to about 20%, about 12% to about 14%, about 12% to about 16%, about 12% to about 18%, about 12% to about 20%, about 14% to about 16%, about 14% to about 18%, about 14% to about 20%, about 16% to about 18%, about 16% to about 20%, or about 18% to about 20% w/w of the composition. In some embodiments, the antioxidant mixture comprises about 5%, about 6%, about 7%, about 8%, about 9%, about 10%, about 12%, about 14%, about 16%, about 18%, or about 20% w/w of the composition. In some embodiments, the antioxidant mixture comprises at least about 5%, about 6%, about 7%, about 8%, about 9%, about 10%, about 12%, about 14%, about 16%, or about 18% w/w of the composition. In some embodiments, the antioxidant mixture comprises at most about 6%, about 7%, about 8%, about 9%, about 10%, about 12%, about 14%, about 16%, about 18%, or about 20% w/w of the composition. In some embodiments, the antioxidant mixture comprises about 20% to about 40% w/w of the composition. In some embodiments, the antioxidant mixture comprises about 20% to about 24%, about 20% to about 28%, about 20% to about 32%, about 20% to about 36%, about 20% to about 40%, about 24% to about 28%, about 24% to about 32%, about 24% to about 36%, about 24% to about 40%, about 28% to about 32%, about 28% to about 36%, about 28% to about 40%, about 32% to about 36%, about 32% to about 40%, or about 36% to about 40% w/w of the composition. In some embodiments, the antioxidant mixture comprises about 20%, about 24%, about 28%, about 32%, about 36%, or about 40% w/w of the composition. In some embodiments, the antioxidant mixture comprises at least about 20%, about 24%, about 28%, about 32%, or about 36% w/w of the composition. In some embodiments, the antioxidant mixture comprises at most about 24%, about 28%, about 32%, about 36%, or about 40% w/w of the composition.

In some embodiments, the composition further includes a hydrator and/or a humectant. In some embodiments, the internal phase further includes the hydrator and/or the humectant. In some embodiments, the external phase further includes the hydrator and/or the humectant. In some embodiments, the internal phase and the external phase both include the hydrator and/or the humectant. In some embodiments, the hydrator and/or humectant includes hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10. in some embodiments, the hydrator and/or humectant promotes moisture in the skin region to which a composition comprising the hydrator and/or humectant is applied.

In some embodiments, the hydrator and/or humectant comprises about 0.001% to about 1% w/w of the composition. In some embodiments, the hydrator and/or humectant comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the hydrator and/or humectant comprises about 0.001%, about 0.002%, about 0.003%, about 0.004%, about 0.005%, about 0.006%, about 0.007%, about 0.008%, about 0.009%, or about 0.01% w/w of the composition. In some embodiments, the hydrator and/or humectant comprises at least about 0.001%, about 0.002%, about 0.003%, about 0.004%, about 0.005%, about 0.006%, about 0.007%, about 0.008%, or about 0.009% w/w of the composition. In some embodiments, the hydrator and/or humectant comprises at most about 0.002%, about 0.003%, about 0.004%, about 0.005%, about 0.006%, about 0.007%, about 0.008%, about 0.009%, or about 0.01% w/w of the composition. In some embodiments, the hydrator and/or humectant comprises about 0.01% to about 0.1% w/w of the composition. In some embodiments, the hydrator and/or humectant comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the hydrator and/or humectant comprises about 0.01%, about 0.02%, about 0.03%, about 0.04%, about 0.05%, about 0.06%, about 0.07%, about 0.08%, about 0.09%, or about 0.1% w/w of the composition. In some embodiments, the hydrator and/or humectant comprises at least about 0.01%, about 0.02%, about 0.03%, about 0.04%, about 0.05%, about 0.06%, about 0.07%, about 0.08%, or about 0.09% w/w of the composition. In some embodiments, the hydrator and/or humectant comprises at most about 0.02%, about 0.03%, about 0.04%, about 0.05%, about 0.06%, about 0.07%, about 0.08%, about 0.09%, or about 0.1% w/w of the composition. In some embodiments, the hydrator and/or humectant comprises about 0.1% to about 1% w/w of the composition. In some embodiments, the hydrator and/or humectant comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the hydrator and/or humectant comprises about 0.1%, about 0.2%, about 0.3%, about 0.4%, about 0.5%, about 0.6%, about 0.7%, about 0.8%, about 0.9%, or about 1% w/w of the composition. In some embodiments, the hydrator and/or humectant comprises at least about 0.1%, about 0.2%, about 0.3%, about 0.4%, about 0.5%, about 0.6%, about 0.7%, about 0.8%, or about 0.9% w/w of the composition. In some embodiments, the hydrator and/or humectant comprises at most about 0.2%, about 0.3%, about 0.4%, about 0.5%, about 0.6%, about 0.7%, about 0.8%, about 0.9%, or about 1% w/w of the composition.

In some embodiments, the external phase is an oil phase. In some embodiments, the external phase is oil-based. In some embodiments, the external phase is a silicone phase. In some embodiments, the external phase is silicone-based. In some embodiments, the external phase includes an antioxidant mixture. In some embodiments, the external phase includes a moisturizer mixture. In some embodiments, the external phase includes both the antioxidant mixture and the moisturizer mixture.

In some embodiments, the external phase comprises about 5% to about 50% w/w of the composition. In some embodiments, the external phase comprises about 5% to about 7.5%, about 5% to about 10%, about 5% to about 12.5%, about 5% to about 15%, about 5% to about 20%, about 5% to about 25%, about 5% to about 30%, about 5% to about 35%, about 5% to about 40%, about 5% to about 45%, about 5% to about 50%, about 7.5% to about 10%, about 7.5% to about 12.5%, about 7.5% to about 15%, about 7.5% to about 20%, about 7.5% to about 25%, about 7.5% to about 30%, about 7.5% to about 35%, about 7.5% to about 40%, about 7.5% to about 45%, about 7.5% to about 50%, about 10% to about 12.5%, about 10% to about 15%, about 10% to about 20%, about 10% to about 25%, about 10% to about 30%, about 10% to about 35%, about 10% to about 40%, about 10% to about 45%, about 10% to about 50%, about 12.5% to about 15%, about 12.5% to about 20%, about 12.5% to about 25%, about 12.5% to about 30%, about 12.5% to about 35%, about 12.5% to about 40%, about 12.5% to about 45%, about 12.5% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50% w/w of the composition. In some embodiments, the external phase comprises about 5%, about 7.5%, about 10%, about 12.5%, about 15%, about 20%, about 25%, about 30%, about 35%, about 40%, about 45%, or about 50% w/w of the composition. In some embodiments, the external phase comprises at least about 5%, about 7.5%, about 10%, about 12.5%, about 15%, about 20%, about 25%, about 30%, about 35%, about 40%, or about 45% w/w of the composition. In some embodiments, the external phase comprises at most about 7.5%, about 10%, about 12.5%, about 15%, about 20%, about 25%, about 30%, about 35%, about 40%, about 45%, or about 50% w/w of the composition. In some embodiments, the external phase comprises about 50% to about 99% w/w of the composition. In some embodiments, the external phase comprises about 50% to about 55%, about 50% to about 60%, about 50% to about 65%, about 50% to about 70%, about 50% to about 80%, about 50% to about 90%, about 50% to about 95%, about 50% to about 99%, about 55% to about 60%, about 55% to about 65%, about 55% to about 70%, about 55% to about 80%, about 55% to about 90%, about 55% to about 95%, about 55% to about 99%, about 60% to about 65%, about 60% to about 70%, about 60% to about 80%, about 60% to about 90%, about 60% to about 95%, about 60% to about 99%, about 65% to about 70%, about 65% to about 80%, about 65% to about 90%, about 65% to about 95%, about 65% to about 99%, about 70% to about 80%, about 70% to about 90%, about 70% to about 95%, about 70% to about 99%, about 80% to about 90%, about 80% to about 95%, about 80% to about 99%, about 90% to about 95%, about 90% to about 99%, or about 95% to about 99% w/w of the composition. In some embodiments, the external phase comprises about 50%, about 55%, about 60%, about 65%, about 70%, about 80%, about 90%, about 95%, or about 99% w/w of the composition. In some embodiments, the external phase comprises at least about 50%, about 55%, about 60%, about 65%, about 70%, about 80%, about 90%, or about 95% w/w of the composition. In some embodiments, the external phase comprises at most about 55%, about 60%, about 65%, about 70%, about 80%, about 90%, about 95%, or about 99% of the composition.

In some embodiments, the composition further includes an antioxidant mixture. In some embodiments, the external phase includes the antioxidant mixture. In some embodiments, the internal phase includes the antioxidant mixture. In some embodiments, the internal phase and the external phase both include the antioxidant mixture. In some embodiments, the antioxidant mixture includes at least one antioxidant. In some embodiments, the at least antioxidant includes dipalmitoyl hydroxyproline. In some embodiments, the at least one antioxidant includes tetrahexyldecyl ascorbate, ubiquinone, vitamin E, or any combination of two or more thereof. In some embodiments, the at least one antioxidant includes dipalmitoyl hydroxyproline, tetrahexyldecylascorbate, ubiquinone, vitamin E, or any combination of two or more thereof. In some embodiments, the antioxidant mixture includes dipalmitoyl hydroxyproline, tetrahexyldecyl ascorbate, ubiquinone, vitamin E, or any combination of two or more thereof. In some embodiments, the at least one antioxidant includes resveratrol, epigallocatechin gallate, ellagic acid, ursolic acid, oleanolic acid, chlorogenic acid, rutin, hesperidin, quercetin, bakuchiol, botanical extracts of any part of a plant including the leaves, flowers, seeds, fruits, stems, barks or roots (e.g., Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract), vitamin or vitamin-like compounds and their derivatives (e.g., tocopherol, tocoopheryl acetate, ubiquinone, ascorbic acid, magnesium ascorbyl phosphate, tetrahexyldecyl ascorbate, retinol, retinaldehyde, retinyl palmitate) or any combination of two or more thereof. In some embodiments, the antioxidant mixture further includes resveratrol, epigallocatechin gallate, ellagic acid, ursolic acid, oleanolic acid, chlorogenic acid, rutin, hesperidin, quercetin, bakuchiol, botanical extracts of any part of a plant including the leaves, flowers, seeds, fruits, stems, barks, or roots (e.g., Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract), vitamin or vitamin-like compounds and their derivatives (e.g., tocopherol, tocoopheryl acetate, ubiquinone, ascorbic acid, magnesium ascorbyl phosphate, tetrahexyldecyl ascorbate, retinol, retinaldehyde, retinyl palmitate) or any combination of two or more thereof.

In some embodiments, the antioxidant mixture comprises about 0.001% to about 8% w/w of the composition. In some embodiments, the antioxidant mixture comprises about 0.001% to about 0.01% w/w of the composition. In some embodiments, the antioxidant mixture comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the antioxidant mixture comprises about 0.001%, about 0.002%, about 0.003%, about 0.004%, about 0.005%, about 0.006%, about 0.007%, about 0.008%, about 0.009%, or about 0.01% w/w of the composition. In some embodiments, the antioxidant mixture comprises at least about 0.001%, about 0.002%, about 0.003%, about 0.004%, about 0.005%, about 0.006%, about 0.007%, about 0.008%, or about 0.009% w/w of the composition. In some embodiments, the antioxidant mixture comprises at most about 0.002%, about 0.003%, about 0.004%, about 0.005%, about 0.006%, about 0.007%, about 0.008%, about 0.009%, or about 0.01% w/w of the composition. In some embodiments, the antioxidant mixture comprises about 0.01% to about 0.1% w/w of the composition. In some embodiments, the antioxidant mixture comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the antioxidant mixture comprises about 0.01%, about 0.02%, about 0.03%, about 0.04%, about 0.05%, about 0.06%, about 0.07%, about 0.08%, about 0.09%, or about 0.1% w/w of the composition. In some embodiments, the antioxidant mixture comprises at least about 0.01%, about 0.02%, about 0.03%, about 0.04%, about 0.05%, about 0.06%, about 0.07%, about 0.08%, or about 0.09% w/w of the composition. In some embodiments, the antioxidant mixture comprises at most about 0.02%, about 0.03%, about 0.04%, about 0.05%, about 0.06%, about 0.07%, about 0.08%, about 0.09%, or about 0.1% w/w of the composition. In some embodiments, the antioxidant mixture comprises about 0.1% to about 1% w/w of the composition. In some embodiments, the antioxidant mixture comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the antioxidant mixture comprises about 0.1%, about 0.2%, about 0.3%, about 0.4%, about 0.5%, about 0.6%, about 0.7%, about 0.8%, about 0.9%, or about 1% w/w of the composition. In some embodiments, the antioxidant mixture comprises at least about 0.1%, about 0.2%, about 0.3%, about 0.4%, about 0.5%, about 0.6%, about 0.7%, about 0.8%, or about 0.9% w/w of the composition. In some embodiments, the antioxidant mixture comprises at most about 0.2%, about 0.3%, about 0.4%, about 0.5%, about 0.6%, about 0.7%, about 0.8%, about 0.9%, or about 1% w/w of the composition. In some embodiments, the antioxidant mixture comprises about 0.1% to about 0.25% w/w of the composition. In some embodiments, the antioxidant mixture comprises about 0.1% to about 0.15%, about 0.1% to about 0.2%, about 0.1% to about 0.25%, about 0.15% to about 0.2%, about 0.15% to about 0.25%, or about 0.2% to about 0.25% w/w of the composition. In some embodiments, the antioxidant mixture comprises about 0.1%, about 0.15%, about 0.2%, or about 0.25% w/w of the composition. In some embodiments, the antioxidant mixture comprises at least about 0.1%, about 0.15%, or about 0.2% w/w of the composition. In some embodiments, the antioxidant mixture comprises at most about 0.15%, about 0.2%, or about 0.25% w/w of the composition. In some embodiments, the antioxidant mixture comprises about 0.01% to about 5% w/w of the composition. In some embodiments, the antioxidant mixture comprises about 0.01% to about 0.5%, about 0.01% to about 1%, about 0.01% to about 1.5%, about 0.01% to about 2%, about 0.01% to about 2.5%, about 0.01% to about 3%, about 0.01% to about 3.5%, about 0.01% to about 4%, about 0.01% to about 4.5%, about 0.01% to about 5%, about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5% w/w of the composition. In some embodiments, the antioxidant mixture comprises about 0.01%, about 0.5%, about 1%, about 1.5%, about 2%, about 2.5%, about 3%, about 3.5%, about 4%, about 4.5%, or about 5% w/w of the composition. In some embodiments, the antioxidant mixture comprises at least about 0.01%, about 0.5%, about 1%, about 1.5%, about 2%, about 2.5%, about 3%, about 3.5%, about 4%, or about 4.5% w/w of the composition. In some embodiments, the antioxidant mixture comprises at most about 0.5%, about 1%, about 1.5%, about 2%, about 2.5%, about 3%, about 3.5%, about 4%, about 4.5%, or about 5% w/w of the composition. In some embodiments, the antioxidant mixture comprises about 5% to about 20% w/w of the composition. In some embodiments, the antioxidant mixture comprises about 5% to about 6%, about 5% to about 7%, about 5% to about 8%, about 5% to about 9%, about 5% to about 10%, about 5% to about 12%, about 5% to about 14%, about 5% to about 16%, about 5% to about 18%, about 5% to about 20%, about 6% to about 7%, about 6% to about 8%, about 6% to about 9%, about 6% to about 10%, about 6% to about 12%, about 6% to about 14%, about 6% to about 16%, about 6% to about 18%, about 6% to about 20%, about 7% to about 8%, about 7% to about 9%, about 7% to about 10%, about 7% to about 12%, about 7% to about 14%, about 7% to about 16%, about 7% to about 18%, about 7% to about 20%, about 8% to about 9%, about 8% to about 10%, about 8% to about 12%, about 8% to about 14%, about 8% to about 16%, about 8% to about 18%, about 8% to about 20%, about 9% to about 10%, about 9% to about 12%, about 9% to about 14%, about 9% to about 16%, about 9% to about 18%, about 9% to about 20%, about 10% to about 12%, about 10% to about 14%, about 10% to about 16%, about 10% to about 18%, about 10% to about 20%, about 12% to about 14%, about 12% to about 16%, about 12% to about 18%, about 12% to about 20%, about 14% to about 16%, about 14% to about 18%, about 14% to about 20%, about 16% to about 18%, about 16% to about 20%, or about 18% to about 20% w/w of the composition. In some embodiments, the antioxidant mixture comprises about 5%, about 6%, about 7%, about 8%, about 9%, about 10%, about 12%, about 14%, about 16%, about 18%, or about 20% w/w of the composition. In some embodiments, the antioxidant mixture comprises at least about 5%, about 6%, about 7%, about 8%, about 9%, about 10%, about 12%, about 14%, about 16%, or about 18% w/w of the composition. In some embodiments, the antioxidant mixture comprises at most about 6%, about 7%, about 8%, about 9%, about 10%, about 12%, about 14%, about 16%, about 18%, or about 20% w/w of the composition. In some embodiments, the antioxidant mixture comprises about 20% to about 40% w/w of the composition. In some embodiments, the antioxidant mixture comprises about 20% to about 24%, about 20% to about 28%, about 20% to about 32%, about 20% to about 36%, about 20% to about 40%, about 24% to about 28%, about 24% to about 32%, about 24% to about 36%, about 24% to about 40%, about 28% to about 32%, about 28% to about 36%, about 28% to about 40%, about 32% to about 36%, about 32% to about 40%, or about 36% to about 40% w/w of the composition. In some embodiments, the antioxidant mixture comprises about 20%, about 24%, about 28%, about 32%, about 36%, or about 40% w/w of the composition. In some embodiments, the antioxidant mixture comprises at least about 20%, about 24%, about 28%, about 32%, or about 36% w/w of the composition. In some embodiments, the antioxidant mixture comprises at most about 24%, about 28%, about 32%, about 36%, or about 40% w/w of the composition.

In some embodiments, the composition further includes a moisturizer mixture. In some embodiments, the external phase includes the moisturizer mixture. In some embodiments, the internal phase includes the moisturizer mixture. In some embodiments, the internal and external phase both include the moisturizer mixture. In some embodiments, the moisturizer mixture includes one or more components for increasing moisture (e.g., one or more emollients) in a skin region to which a composition including the moisturizer mixture is applied. The one or more components may include natural oils, lipids, butters and triglycerides as well as synthetic esters and their derivatives. In some embodiments, the one or more components may include shea butter, jojoba esters, dimethicone, Helianthus annuus (sunflower) seed wax, Helianthus annuus (sunflower) seed oil, Simmondsia chinensis (jojoba) seed oil, Olea europaea (olive) oil, Glycine soja (soybean) oil, Butyrospermum parkii (shea) butter, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, jojoba esters, cetyl esters, or any combination of two or more thereof. In some embodiments, the moisturizer mixture restores a barrier of the skin region. In some embodiments, the barrier is a skin barrier, a mucosal barrier, or any combination of two or more thereof. In some embodiments, the moisturizer mixture promotes reduced redness of a skin region to which the composition is applied.

In some embodiments, the moisturizer mixture comprises about 0.001% to about 80% w/w of the composition. In some embodiments, the moisturizer mixture comprises about 0.001% to about 0.01% w/w of the composition. In some embodiments, the moisturizer mixture comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the moisturizer mixture comprises about 0.001%, about 0.002%, about 0.003%, about 0.004%, about 0.005%, about 0.006%, about 0.007%, about 0.008%, about 0.009%, or about 0.01% w/w of the composition. In some embodiments, the moisturizer mixture comprises at least about 0.001%, about 0.002%, about 0.003%, about 0.004%, about 0.005%, about 0.006%, about 0.007%, about 0.008%, or about 0.009% w/w of the composition. In some embodiments, the moisturizer mixture comprises at most about 0.002%, about 0.003%, about 0.004%, about 0.005%, about 0.006%, about 0.007%, about 0.008%, about 0.009%, or about 0.01% w/w of the composition. In some embodiments, the moisturizer mixture comprises about 0.01% to about 0.1% w/w of the composition. In some embodiments, the moisturizer mixture comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the moisturizer mixture comprises about 0.01%, about 0.02%, about 0.03%, about 0.04%, about 0.05%, about 0.06%, about 0.07%, about 0.08%, about 0.09%, or about 0.1% w/w of the composition. In some embodiments, the moisturizer mixture comprises at least about 0.01%, about 0.02%, about 0.03%, about 0.04%, about 0.05%, about 0.06%, about 0.07%, about 0.08%, or about 0.09% w/w of the composition. In some embodiments, the moisturizer mixture comprises at most about 0.02%, about 0.03%, about 0.04%, about 0.05%, about 0.06%, about 0.07%, about 0.08%, about 0.09%, or about 0.1% w/w of the composition. In some embodiments, the moisturizer mixture comprises about 0.1% to about 1% w/w of the composition. In some embodiments, the moisturizer mixture comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the moisturizer mixture comprises about 0.1%, about 0.2%, about 0.3%, about 0.4%, about 0.5%, about 0.6%, about 0.7%, about 0.8%, about 0.9%, or about 1% w/w of the composition. In some embodiments, the moisturizer mixture comprises at least about 0.1%, about 0.2%, about 0.3%, about 0.4%, about 0.5%, about 0.6%, about 0.7%, about 0.8%, or about 0.9% w/w of the composition. In some embodiments, the moisturizer mixture comprises at most about 0.2%, about 0.3%, about 0.4%, about 0.5%, about 0.6%, about 0.7%, about 0.8%, about 0.9%, or about 1% w/w of the composition. In some embodiments, the moisturizer mixture comprises about 1% to about 5% w/w of the composition. In some embodiments, the moisturizer mixture comprises about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5% w/w of the composition. In some embodiments, the moisturizer mixture comprises about 1%, about 1.5%, about 2%, about 2.5%, about 3%, about 3.5%, about 4%, about 4.5%, or about 5% w/w of the composition. In some embodiments, the moisturizer mixture comprises at least about 1%, about 1.5%, about 2%, about 2.5%, about 3%, about 3.5%, about 4%, or about 4.5% w/w of the composition. In some embodiments, the moisturizer mixture comprises at most about 1.5%, about 2%, about 2.5%, about 3%, about 3.5%, about 4%, about 4.5%, or about 5% w/w of the composition. In some embodiments, the moisturizer mixture comprises about 0.25% to about 25% w/w of the composition. In some embodiments, the moisturizer mixture comprises about 0.25% to about 0.5%, about 0.25% to about 1%, about 0.25% to about 3%, about 0.25% to about 5%, about 0.25% to about 7.5%, about 0.25% to about 10%, about 0.25% to about 12.5%, about 0.25% to about 15%, about 0.25% to about 20%, about 0.25% to about 25%, about 0.5% to about 1%, about 0.5% to about 3%, about 0.5% to about 5%, about 0.5% to about 7.5%, about 0.5% to about 10%, about 0.5% to about 12.5%, about 0.5% to about 15%, about 0.5% to about 20%, about 0.5% to about 25%, about 1% to about 3%, about 1% to about 5%, about 1% to about 7.5%, about 1% to about 10%, about 1% to about 12.5%, about 1% to about 15%, about 1% to about 20%, about 1% to about 25%, about 3% to about 5%, about 3% to about 7.5%, about 3% to about 10%, about 3% to about 12.5%, about 3% to about 15%, about 3% to about 20%, about 3% to about 25%, about 5% to about 7.5%, about 5% to about 10%, about 5% to about 12.5%, about 5% to about 15%, about 5% to about 20%, about 5% to about 25%, about 7.5% to about 10%, about 7.5% to about 12.5%, about 7.5% to about 15%, about 7.5% to about 20%, about 7.5% to about 25%, about 10% to about 12.5%, about 10% to about 15%, about 10% to about 20%, about 10% to about 25%, about 12.5% to about 15%, about 12.5% to about 20%, about 12.5% to about 25%, about 15% to about 20%, about 15% to about 25%, or about 20% to about 25% w/w of the composition. In some embodiments, the moisturizer mixture comprises about 0.25%, about 0.5%, about 1%, about 3%, about 5%, about 7.5%, about 10%, about 12.5%, about 15%, about 20%, or about 25% w/w of the composition. In some embodiments, the moisturizer mixture comprises at least about 0.25%, about 0.5%, about 1%, about 3%, about 5%, about 7.5%, about 10%, about 12.5%, about 15%, or about 20% w/w of the composition. In some embodiments, the moisturizer mixture comprises at most about 0.5%, about 1%, about 3%, about 5%, about 7.5%, about 10%, about 12.5%, about 15%, about 20%, or about 25% w/w of the composition. In some embodiments, the moisturizer mixture comprises about 25% to about 35% w/w of the composition. In some embodiments, the moisturizer mixture comprises about 25% to about 27%, about 25% to about 29%, about 25% to about 31%, about 25% to about 33%, about 25% to about 35%, about 27% to about 29%, about 27% to about 31%, about 27% to about 33%, about 27% to about 35%, about 29% to about 31%, about 29% to about 33%, about 29% to about 35%, about 31% to about 33%, about 31% to about 35%, or about 33% to about 35% w/w of the composition. In some embodiments, the moisturizer mixture comprises about 25%, about 27%, about 29%, about 31%, about 33%, or about 35% w/w of the composition. In some embodiments, the moisturizer mixture comprises at least about 25%, about 27%, about 29%, about 31%, or about 33% w/w of the composition. In some embodiments, the moisturizer mixture comprises at most about 27%, about 29%, about 31%, about 33%, or about 35% w/w of the composition. In some embodiments, the moisturizer mixture comprises about 35% to about 50% w/w of the composition. In some embodiments, the moisturizer mixture comprises about 35% to about 38%, about 35% to about 41%, about 35% to about 44%, about 35% to about 47%, about 35% to about 50%, about 38% to about 41%, about 38% to about 44%, about 38% to about 47%, about 38% to about 50%, about 41% to about 44%, about 41% to about 47%, about 41% to about 50%, about 44% to about 47%, about 44% to about 50%, or about 47% to about 50% w/w of the composition. In some embodiments, the moisturizer mixture comprises about 35%, about 38%, about 41%, about 44%, about 47%, or about 50% w/w of the composition. In some embodiments, the moisturizer mixture comprises at least about 35%, about 38%, about 41%, about 44%, or about 47% w/w of the composition. In some embodiments, the moisturizer mixture comprises at most about 38%, about 41%, about 44%, about 47%, or about 50% w/w of the composition. In some embodiments, the moisturizer mixture comprises about 50% to about 80% w/w of the composition. In some embodiments, the moisturizer mixture comprises about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or about 75% to about 80% w/w of the composition. In some embodiments, the moisturizer mixture comprises about 50%, about 55%, about 60%, about 65%, about 70%, about 75%, or about 80% w/w of the composition. In some embodiments, the moisturizer mixture comprises at least about 50%, about 55%, about 60%, about 65%, about 70%, or about 75% w/w of the composition. In some embodiments, the moisturizer mixture comprises at most about 55%, about 60%, about 65%, about 70%, about 75%, or about 80% w/w of the composition.

In some embodiments, the composition further includes a carbohydrate. In some embodiments, the external phase includes the carbohydrate. In some embodiments, the internal phase includes the carbohydrate. In some embodiments, the internal phase and the external phase both include the carbohydrate. In some embodiments, the carbohydrate comprises a viscosity agent and/or a moisturizing agent. In some embodiments, the carbohydrate is water soluble or water miscible. In some embodiment, the carbohydrate is biosaccharide gum-1. In some embodiments, the skin region to which a composition comprising the carbohydrate is applied has an improved level of moisture relative to a level of moisture of a skin region to which a composition comprising the carbohydrate is not applied.

In some embodiments, the carbohydrate comprises about 0.001% to about 5% w/w of the composition. In some embodiments, the carbohydrate comprises about 0.001% to about 0.01% w/w of the composition. In some embodiments, the carbohydrate comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the carbohydrate comprises about 0.001%, about 0.002%, about 0.003%, about 0.004%, about 0.005%, about 0.006%, about 0.007%, about 0.008%, about 0.009%, or about 0.01% w/w of the composition. In some embodiments, the carbohydrate comprises at least about 0.001%, about 0.002%, about 0.003%, about 0.004%, about 0.005%, about 0.006%, about 0.007%, about 0.008%, or about 0.009% w/w of the composition. In some embodiments, the carbohydrate comprises at most about 0.002%, about 0.003%, about 0.004%, about 0.005%, about 0.006%, about 0.007%, about 0.008%, about 0.009%, or about 0.01% w/w of the composition. In some embodiments, the carbohydrate comprises about 0.01% to about 0.1% w/w of the composition. In some embodiments, the carbohydrate comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the carbohydrate comprises about 0.01%, about 0.02%, about 0.03%, about 0.04%, about 0.05%, about 0.06%, about 0.07%, about 0.08%, about 0.09%, or about 0.1% w/w of the composition. In some embodiments, the carbohydrate comprises at least about 0.01%, about 0.02%, about 0.03%, about 0.04%, about 0.05%, about 0.06%, about 0.07%, about 0.08%, or about 0.09% w/w of the composition. In some embodiments, the carbohydrate comprises at most about 0.02%, about 0.03%, about 0.04%, about 0.05%, about 0.06%, about 0.07%, about 0.08%, about 0.09%, or about 0.1% w/w of the composition. In some embodiments, the carbohydrate comprises about 0.1% to about 1% w/w of the composition. In some embodiments, the carbohydrate comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the carbohydrate comprises about 0.1%, about 0.2%, about 0.3%, about 0.4%, about 0.5%, about 0.6%, about 0.7%, about 0.8%, about 0.9%, or about 1% w/w of the composition. In some embodiments, the carbohydrate comprises at least about 0.1%, about 0.2%, about 0.3%, about 0.4%, about 0.5%, about 0.6%, about 0.7%, about 0.8%, or about 0.9% w/w of the composition. In some embodiments, the carbohydrate comprises at most about 0.2%, about 0.3%, about 0.4%, about 0.5%, about 0.6%, about 0.7%, about 0.8%, about 0.9%, or about 1% w/w of the composition. In some embodiments, the carbohydrate comprises about 1% to about 5% w/w of the composition. In some embodiments, the carbohydrate comprises about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5% w/w of the composition. In some embodiments, the carbohydrate comprises about 1%, about 1.5%, about 2%, about 2.5%, about 3%, about 3.5%, about 4%, about 4.5%, or about 5% w/w of the composition. In some embodiments, the carbohydrate comprises at least about 1%, about 1.5%, about 2%, about 2.5%, about 3%, about 3.5%, about 4%, or about 4.5% w/w of the composition. In some embodiments, the carbohydrate comprises at most about 1.5%, about 2%, about 2.5%, about 3%, about 3.5%, about 4%, about 4.5%, or about 5% w/w of the composition.

In some embodiments, the composition includes a second antioxidant. In some embodiments, the external phase includes the second antioxidant. In some embodiments, the internal phase includes the second antioxidant. In some embodiments, the internal phase and the external phase both include the second antioxidant. In some embodiments, the second antioxidant includes dipalmitoyl hydroxyproline. In some embodiments, dipalmitoyl hydroxyproline promotes increased firmness of a skin region and promotes collagen synthesis in the skin region when included in the compositions herein, which is an unexpected characteristic of an antioxidant. In some embodiments, the second antioxidant increases firmness of a skin region to which the composition including the second antioxidant is applied. In some embodiments, the second antioxidant increases smoothness of a skin region to which the composition including the second antioxidant is applied. In some embodiments, the second antioxidant promotes collagen synthesis in the skin region.

In some embodiments, the second antioxidant comprises about 0.001% to about 1% w/w of the composition. In some embodiments, the second antioxidant comprises about 0.001% to about 0.01% w/w of the composition. In some embodiments, the second antioxidant comprises about 0.001% to about 0.002%, about 0.001% to about 0.003%, about 0.001% to about 0.004%, about 0.001% to about 0.005%, about 0.001% to about 0.006%, about 0.001% to about 0.007%, about 0.001% to about 0.008%, about 0.001% to about 0.009%, about 0.001% to about 0.01%, about 0.002% to about 0.003%, about 0.002% to about 0.004%, about 0.002% to about 0.005%, about 0.002% to about 0.006%, about 0.002% to about 0.007%, about 0.002% to about 0.008%, about 0.002% to about 0.009%, about 0.002% to about 0.01%, about 0.003% to about 0.004%, about 0.003% to about 0.005%, about 0.003% to about 0.006%, about 0.003% to about 0.007%, about 0.003% to about 0.008%, about 0.003% to about 0.009%, about 0.003% to about 0.01%, about 0.004% to about 0.005%, about 0.004% to about 0.006%, about 0.004% to about 0.007%, about 0.004% to about 0.008%, about 0.004% to about 0.009%, about 0.004% to about 0.01%, about 0.005% to about 0.006%, about 0.005% to about 0.007%, about 0.005% to about 0.008%, about 0.005% to about 0.009%, about 0.005% to about 0.01%, about 0.006% to about 0.007%, about 0.006% to about 0.008%, about 0.006% to about 0.009%, about 0.006% to about 0.01%, about 0.007% to about 0.008%, about 0.007% to about 0.009%, about 0.007% to about 0.01%, about 0.008% to about 0.009%, about 0.008% to about 0.01%, or about 0.009% to about 0.01% w/w of the composition. In some embodiments, the second antioxidant comprises about 0.001%, about 0.002%, about 0.003%, about 0.004%, about 0.005%, about 0.006%, about 0.007%, about 0.008%, about 0.009%, or about 0.01% w/w of the composition. In some embodiments, the second antioxidant comprises at least about 0.001%, about 0.002%, about 0.003%, about 0.004%, about 0.005%, about 0.006%, about 0.007%, about 0.008%, or about 0.009% w/w of the composition. In some embodiments, the second antioxidant comprises at most about 0.002%, about 0.003%, about 0.004%, about 0.005%, about 0.006%, about 0.007%, about 0.008%, about 0.009%, or about 0.01% w/w of the composition. In some embodiments, the second antioxidant comprises about 0.01% to about 0.1% w/w of the composition. In some embodiments, the second antioxidant comprises about 0.01% to about 0.02%, about 0.01% to about 0.03%, about 0.01% to about 0.04%, about 0.01% to about 0.05%, about 0.01% to about 0.06%, about 0.01% to about 0.07%, about 0.01% to about 0.08%, about 0.01% to about 0.09%, about 0.01% to about 0.1%, about 0.02% to about 0.03%, about 0.02% to about 0.04%, about 0.02% to about 0.05%, about 0.02% to about 0.06%, about 0.02% to about 0.07%, about 0.02% to about 0.08%, about 0.02% to about 0.09%, about 0.02% to about 0.1%, about 0.03% to about 0.04%, about 0.03% to about 0.05%, about 0.03% to about 0.06%, about 0.03% to about 0.07%, about 0.03% to about 0.08%, about 0.03% to about 0.09%, about 0.03% to about 0.1%, about 0.04% to about 0.05%, about 0.04% to about 0.06%, about 0.04% to about 0.07%, about 0.04% to about 0.08%, about 0.04% to about 0.09%, about 0.04% to about 0.1%, about 0.05% to about 0.06%, about 0.05% to about 0.07%, about 0.05% to about 0.08%, about 0.05% to about 0.09%, about 0.05% to about 0.1%, about 0.06% to about 0.07%, about 0.06% to about 0.08%, about 0.06% to about 0.09%, about 0.06% to about 0.1%, about 0.07% to about 0.08%, about 0.07% to about 0.09%, about 0.07% to about 0.1%, about 0.08% to about 0.09%, about 0.08% to about 0.1%, or about 0.09% to about 0.1% w/w of the composition. In some embodiments, the second antioxidant comprises about 0.01%, about 0.02%, about 0.03%, about 0.04%, about 0.05%, about 0.06%, about 0.07%, about 0.08%, about 0.09%, or about 0.1% w/w of the composition. In some embodiments, the second antioxidant comprises at least about 0.01%, about 0.02%, about 0.03%, about 0.04%, about 0.05%, about 0.06%, about 0.07%, about 0.08%, or about 0.09% w/w of the composition. In some embodiments, the second antioxidant comprises at most about 0.02%, about 0.03%, about 0.04%, about 0.05%, about 0.06%, about 0.07%, about 0.08%, about 0.09%, or about 0.1% w/w of the composition. In some embodiments, the second antioxidant comprises about 0.1% to about 1% w/w of the composition. In some embodiments, the second antioxidant comprises about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the composition. In some embodiments, the second antioxidant comprises about 0.1%, about 0.2%, about 0.3%, about 0.4%, about 0.5%, about 0.6%, about 0.7%, about 0.8%, about 0.9%, or about 1% w/w of the composition. In some embodiments, the second antioxidant comprises at least about 0.1%, about 0.2%, about 0.3%, about 0.4%, about 0.5%, about 0.6%, about 0.7%, about 0.8%, or about 0.9% w/w of the composition. In some embodiments, the second antioxidant comprises at most about 0.2%, about 0.3%, about 0.4%, about 0.5%, about 0.6%, about 0.7%, about 0.8%, about 0.9%, or about 1% w/w of the composition.

In some embodiments, the composition further comprises one or more excipients. The one or more excipients may include one or more emulsifiers. The one or more excipients may include one or more emollients. The one or more excipients may also include one of more pH adjustors, one or more preservatives, and one or more chelating agents. The one or more pH adjustors may include sodium hydroxide, ammonium hydroxide, triethanolamine, aminomethyl propanol, citric acid, or any combination of two or more thereof. The one or more preservatives may include phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, or any combination of two or more thereof. The one or more chelating agents may include tetrasodium glutamate diacetate, disodium EDTA, tetrasodium EDTA, etidronic acid, trisodium dicarboxymethyl alaninate, trisodium ethylenediamine disuccinate, sodium phytate, or any combination of two or more thereof. The one or more excipients may be included in the internal phase, the external phase, or both. In some embodiments, the excipients may also include palmitic acid, caprylic/capric triglyceride, glycerin, squalane, dimethicone, polyglyceryl-6 polyhydroxystearate, glyceryl behenate/eicosadioate, polyglyceryl-6 polyricinoleate, magnesium sulfate, or any combination of two or more thereof.

In some embodiments, the one or more excipients comprises about 1% to about 80% w/w of the composition. In some embodiments, the one or more excipients comprises about 1% to about 10% w/w of the composition. In some embodiments, the one or more excipients comprises about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% w/w of the composition. In some embodiments, the one or more excipients comprises about 1%, about 2%, about 3%, about 4%, about 5%, about 6%, about 7%, about 8%, about 9%, or about 10% w/w of the composition. In some embodiments, the one or more excipients comprises at least about 1%, about 2%, about 3%, about 4%, about 5%, about 6%, about 7%, about 8%, or about 9% w/w of the composition. In some embodiments, the one or more excipients comprises at most about 2%, about 3%, about 4%, about 5%, about 6%, about 7%, about 8%, about 9%, or about 10% w/w of the composition. In some embodiments, the one or more excipients comprises about 10% to about 30% w/w of the composition. In some embodiments, the one or more excipients comprises about 10% to about 11%, about 10% to about 12%, about 10% to about 13%, about 10% to about 14%, about 10% to about 15%, about 10% to about 17.5%, about 10% to about 20%, about 10% to about 22.5%, about 10% to about 25%, about 10% to about 30%, about 11% to about 12%, about 11% to about 13%, about 11% to about 14%, about 11% to about 15%, about 11% to about 17.5%, about 11% to about 20%, about 11% to about 22.5%, about 11% to about 25%, about 11% to about 30%, about 12% to about 13%, about 12% to about 14%, about 12% to about 15%, about 12% to about 17.5%, about 12% to about 20%, about 12% to about 22.5%, about 12% to about 25%, about 12% to about 30%, about 13% to about 14%, about 13% to about 15%, about 13% to about 17.5%, about 13% to about 20%, about 13% to about 22.5%, about 13% to about 25%, about 13% to about 30%, about 14% to about 15%, about 14% to about 17.5%, about 14% to about 20%, about 14% to about 22.5%, about 14% to about 25%, about 14% to about 30%, about 15% to about 17.5%, about 15% to about 20%, about 15% to about 22.5%, about 15% to about 25%, about 15% to about 30%, about 17.5% to about 20%, about 17.5% to about 22.5%, about 17.5% to about 25%, about 17.5% to about 30%, about 20% to about 22.5%, about 20% to about 25%, about 20% to about 30%, about 22.5% to about 25%, about 22.5% to about 30%, or about 25% to about 30% w/w of the composition. In some embodiments, the one or more excipients comprises about 10%, about 11%, about 12%, about 13%, about 14%, about 15%, about 17.5%, about 20%, about 22.5%, about 25%, or about 30% w/w of the composition. In some embodiments, the one or more excipients comprises at least about 10%, about 11%, about 12%, about 13%, about 14%, about 15%, about 17.5%, about 20%, about 22.5%, or about 25% w/w of the composition. In some embodiments, the one or more excipients comprises at most about 11%, about 12%, about 13%, about 14%, about 15%, about 17.5%, about 20%, about 22.5%, about 25%, or about 30% w/w of the composition. In some embodiments, the one or more excipients comprises about 30% to about 80% w/w of the composition. In some embodiments, the one or more excipients comprises about 30% to about 35%, about 30% to about 40%, about 30% to about 45%, about 30% to about 50%, about 30% to about 60%, about 30% to about 70%, about 30% to about 80%, about 35% to about 40%, about 35% to about 45%, about 35% to about 50%, about 35% to about 60%, about 35% to about 70%, about 35% to about 80%, about 40% to about 45%, about 40% to about 50%, about 40% to about 60%, about 40% to about 70%, about 40% to about 80%, about 45% to about 50%, about 45% to about 60%, about 45% to about 70%, about 45% to about 80%, about 50% to about 60%, about 50% to about 70%, about 50% to about 80%, about 60% to about 70%, about 60% to about 80%, or about 70% to about 80% w/w of the composition. In some embodiments, the one or more excipients comprises about 30%, about 35%, about 40%, about 45%, about 50%, about 60%, about 70%, or about 80% w/w of the composition. In some embodiments, the one or more excipients comprises at least about 30%, about 35%, about 40%, about 45%, about 50%, about 60%, or about 70% w/w of the composition. In some embodiments, the one or more excipients comprises at most about 35%, about 40%, about 45%, about 50%, about 60%, about 70%, or about 80% w/w of the composition.

In some embodiments, the pH of the internal phase, the external phase, or the composition as a whole may have a pH of about 3.5 to about 6.5. In some embodiments, the pH is about 3.5 to about 6.5. In some embodiments, the pH is about 3.5 to about 3.75, about 3.5 to about 4, about 3.5 to about 4.25, about 3.5 to about 4.5, about 3.5 to about 4.75, about 3.5 to about 5, about 3.5 to about 5.25, about 3.5 to about 5.5, about 3.5 to about 5.75, about 3.5 to about 6, about 3.5 to about 6.5, about 3.75 to about 4, about 3.75 to about 4.25, about 3.75 to about 4.5, about 3.75 to about 4.75, about 3.75 to about 5, about 3.75 to about 5.25, about 3.75 to about 5.5, about 3.75 to about 5.75, about 3.75 to about 6, about 3.75 to about 6.5, about 4 to about 4.25, about 4 to about 4.5, about 4 to about 4.75, about 4 to about 5, about 4 to about 5.25, about 4 to about 5.5, about 4 to about 5.75, about 4 to about 6, about 4 to about 6.5, about 4.25 to about 4.5, about 4.25 to about 4.75, about 4.25 to about 5, about 4.25 to about 5.25, about 4.25 to about 5.5, about 4.25 to about 5.75, about 4.25 to about 6, about 4.25 to about 6.5, about 4.5 to about 4.75, about 4.5 to about 5, about 4.5 to about 5.25, about 4.5 to about 5.5, about 4.5 to about 5.75, about 4.5 to about 6, about 4.5 to about 6.5, about 4.75 to about 5, about 4.75 to about 5.25, about 4.75 to about 5.5, about 4.75 to about 5.75, about 4.75 to about 6, about 4.75 to about 6.5, about 5 to about 5.25, about 5 to about 5.5, about 5 to about 5.75, about 5 to about 6, about 5 to about 6.5, about 5.25 to about 5.5, about 5.25 to about 5.75, about 5.25 to about 6, about 5.25 to about 6.5, about 5.5 to about 5.75, about 5.5 to about 6, about 5.5 to about 6.5, about 5.75 to about 6, about 5.75 to about 6.5, or about 6 to about 6.5. In some embodiments, the pH is about 3.5, about 3.75, about 4, about 4.25, about 4.5, about 4.75, about 5, about 5.25, about 5.5, about 5.75, about 6, or about 6.5. In some embodiments, the pH is at least about 3.5, about 3.75, about 4, about 4.25, about 4.5, about 4.75, about 5, about 5.25, about 5.5, about 5.75, or about 6. In some embodiments, the pH is at most about 3.75, about 4, about 4.25, about 4.5, about 4.75, about 5, about 5.25, about 5.5, about 5.75, about 6, or about 6.5.

In some embodiments, the composition is a formulation. In some embodiments, the formulation is a cream, lotion, serum, or solid stick. In some embodiments, the composition may be formulated to be applied to a skin region. In some embodiments, the skin region may be the skin region of a mammal. In some embodiments, the mammal may be a human. In some embodiments, the human may be female. In some embodiments, the skin region may be vulvar or related to the vulva. In some embodiments, the human may be male. In some embodiments, the skin region may be penile, scrotal, or related to the penis or the scrotum.

In some embodiments, the composition may be formulated to be applied externally to a skin region. In some embodiments, the skin region may be of the face, neck, abdomen, chest, arms, legs, vulva, penis, scrotum, or any combination of two or more thereof. In some embodiments, the skin region may be of the vulva. In some embodiments, the skin region may be of the penis, scrotum, or both. In some embodiments, the composition may be formulated as a lotion, cream, serum, solid-stick, or other formulation. While a lotion, cream, serum, and solid-stick are listed as formulations, these are exemplary and the composition may be formulated as another formulation. In some embodiments, the composition may be formulated for topical administration to the skin region. In some embodiments, after the composition is applied to the skin region, the skin region may have an improved level of moisture relative to a reference level of moisture. In some embodiments, the reference level of moisture is a level of moisture of the skin region before the composition was applied to the skin region. In some embodiments, the level of moisture is a predetermined level of moisture, for example, a level of moisture resulting in dry skin. In some embodiments, after the composition is applied, the skin region may have an improved (e.g., reduced) level of redness relative to a reference redness of the skin region. In some embodiments, the reference level of redness is a level of redness of the skin region before the composition was applied to the skin region. In some embodiments, the level of redness is a predetermined level of redness, for example, a redness of the skin region. In some embodiments, the human may be experiencing symptoms of condition or disorder. In some embodiments, the symptoms may be menopausal symptoms, perimenopausal symptoms, premenopausal symptoms, or postmenopausal symptoms. In some embodiments, the symptoms may be dryness, irritation, itch, or redness of the skin region. While certain symptoms are listed, these symptoms are exemplary and the human may be experiencing other symptoms. In some embodiments, the symptoms are reduced after external application of the composition.

### Methods

In an aspect, provided herein, is a method of applying a composition described herein to a skin region to improve one or more aspects of skin health.

The improvement of one or more aspects will depend upon the active ingredient(s) contained within the composition. In some embodiments, the one or more aspects of skin health may be a level of moisture in the skin region, a redness of the skin region, or one or more symptoms of a condition, such as menopause. In some embodiments, the symptoms may include dryness, irritation, rash, redness, or other symptoms. While certain symptoms are listed, these symptoms are exemplary, and other symptoms may be improved.

In some embodiments, the composition is externally administered to the skin region. In some embodiments, the composition may be formulated for topical administration, where being externally administered is being administered topically. In some embodiments, the composition may be formulated as a cream, lotion, serum, or solid-stick that is externally or topically administered.

In some embodiments, the skin region is of the face, neck, abdomen, chest, arms, legs, vulva, penis, scrotum, or any combination of two or more thereof. In some embodiments, the skin region may be of the vulva. In some embodiments, the skin region may be of the penis, scrotum, or both.

In some embodiments, one or more aspects of the skin region may be maintained or improved after external administration of the composition. For example, the skin region may be soothed after external administration of the application. As referred to herein, “soothed” refers to having one or more symptoms reduced. For example, a level of burning of the vulva after externally administering the composition is improved (e.g., reduced) relative to a level of burning of the vulva immediately after shaving or prior to shaving. As another example, a level of itching of the vulva after externally administering the composition is improved (e.g., reduced) relative to a level of itching of the vulva immediately after shaving or prior to shaving. As yet another example, a level of stinging of the vulva after externally administering the composition is improved (e.g., reduced) relative to a level of stinging of the vulva immediately after shaving or prior to shaving.

In some embodiments, the skin region may be of a mammal. In some embodiments, the mammal may be a human. In some embodiments, the human may be female. In some embodiments, the skin region may be of the vulva of the human female. In some embodiments, the composition is externally applied to the vulva after a portion or all of the vulva has been shaved. In some embodiments, the composition is externally applied to the vulva within 30 minutes, 1 hour, 2 hours, 3 hours, 4 hours, 5 hours, 6 hours, 7 hours, 8 hours, 9 hours, 10 hours, 12 hours, 15 hours, 18 hours, or 24 hours of shaving. In some embodiments, a level of irritation of the vulva after externally administering the composition is improved (e.g., reduced) relative to a level of irritation of the vulva immediately after shaving. In some embodiments, the vulva is soothed after externally administering the composition after shaving. In some embodiments, a texture of the vulva after externally administering the composition is improved relative to a texture of the vulva immediately after shaving or prior to shaving. For example, the softness, smoothness, and/or plumpness of the vulva may be improved after externally administering the composition. In some embodiments, a redness of the vulva after externally administering the composition is improved relative to a redness of the vulva after shaving or before the composition is externally administered. In some embodiments, a level of moisture of the vulva after externally administering the composition is increased relative to a level of moisture of the vulva after shaving or before externally administering the composition.

In some embodiments, the skin region may be of a mammal. In some embodiments, the mammal may be a human. In some embodiments, the human may be male. In some embodiments, the skin region may include the external genital area of the human male (e.g., an area of skin where hair (e.g., pubic hair) grows and/or has grown). In some embodiments, the skin region may be of the penis and/or scrotum of the human male. In some embodiments, the composition is externally applied to the penis and/or scrotum after a portion or all of the penis and/or scrotum has been shaved. In some embodiments, the composition is externally applied to the penis and/or scrotum within 30 minutes, 1 hour, 2 hours, 3 hours, 4 hours, 5 hours, 6 hours, 7 hours, 8 hours, 9 hours, 10 hours, 12 hours, 15 hours, 18 hours, or 24 hours of shaving. In some embodiments, a level of irritation of the penis and/or scrotum after externally administering the composition is improved (e.g., reduced) relative to a level of irritation of the penis and/or scrotum immediately after shaving. In some embodiments, the penis and/or scrotum is soothed after externally administering the composition after shaving. In some embodiments, a texture of the penis and/or scrotum after externally administering the composition is improved relative to a texture of the penis and/or scrotum immediately after shaving or prior to shaving. For example, the softness, smoothness, and/or plumpness of the penis and/or scrotum may be improved after externally administering the composition. In some embodiments, a redness of the penis and/or scrotum after externally administering the composition is improved relative to a redness of the penis and/or scrotum after shaving or before the composition is externally administered. In some embodiments, a level of moisture of the penis and/or scrotum after externally administering the composition is increased relative to a level of moisture of the penis and/or scrotum after shaving or before externally administering the composition.

In some embodiments, the composition may be administered at a dosage and/or frequency. The dosage and/or frequency may vary based on a health of the skin region and/or symptoms experienced by the skin region.

In some embodiments, the dosage of the composition administered about 0.1 grams to about 0.5 grams. In some embodiments, the dosage of the composition administered about 0.1 grams to about 0.15 grams, about 0.1 grams to about 0.2 grams, about 0.1 grams to about 0.25 grams, about 0.1 grams to about 0.3 grams, about 0.1 grams to about 0.35 grams, about 0.1 grams to about 0.4 grams, about 0.1 grams to about 0.45 grams, about 0.1 grams to about 0.5 grams, about 0.15 grams to about 0.2 grams, about 0.15 grams to about 0.25 grams, about 0.15 grams to about 0.3 grams, about 0.15 grams to about 0.35 grams, about 0.15 grams to about 0.4 grams, about 0.15 grams to about 0.45 grams, about 0.15 grams to about 0.5 grams, about 0.2 grams to about 0.25 grams, about 0.2 grams to about 0.3 grams, about 0.2 grams to about 0.35 grams, about 0.2 grams to about 0.4 grams, about 0.2 grams to about 0.45 grams, about 0.2 grams to about 0.5 grams, about 0.25 grams to about 0.3 grams, about 0.25 grams to about 0.35 grams, about 0.25 grams to about 0.4 grams, about 0.25 grams to about 0.45 grams, about 0.25 grams to about 0.5 grams, about 0.3 grams to about 0.35 grams, about 0.3 grams to about 0.4 grams, about 0.3 grams to about 0.45 grams, about 0.3 grams to about 0.5 grams, about 0.35 grams to about 0.4 grams, about 0.35 grams to about 0.45 grams, about 0.35 grams to about 0.5 grams, about 0.4 grams to about 0.45 grams, about 0.4 grams to about 0.5 grams, or about 0.45 grams to about 0.5 grams. In some embodiments, the dosage of the composition administered about 0.1 grams, about 0.15 grams, about 0.2 grams, about 0.25 grams, about 0.3 grams, about 0.35 grams, about 0.4 grams, about 0.45 grams, or about 0.5 grams. In some embodiments, the dosage of the composition administered at least about 0.1 grams, about 0.15 grams, about 0.2 grams, about 0.25 grams, about 0.3 grams, about 0.35 grams, about 0.4 grams, or about 0.45 grams. In some embodiments, the dosage of the composition administered at most about 0.15 grams, about 0.2 grams, about 0.25 grams, about 0.3 grams, about 0.35 grams, about 0.4 grams, about 0.45 grams, or about 0.5 grams. In some embodiments, the dosage of the composition administered about 0.5 grams to about 1 gram. In some embodiments, the dosage of the composition administered about 0.5 grams to about 0.6 grams, about 0.5 grams to about 0.7 grams, about 0.5 grams to about 0.8 grams, about 0.5 grams to about 0.9 grams, about 0.5 grams to about 1 gram, about 0.6 grams to about 0.7 grams, about 0.6 grams to about 0.8 grams, about 0.6 grams to about 0.9 grams, about 0.6 grams to about 1 gram, about 0.7 grams to about 0.8 grams, about 0.7 grams to about 0.9 grams, about 0.7 grams to about 1 gram, about 0.8 grams to about 0.9 grams, about 0.8 grams to about 1 gram, or about 0.9 grams to about 1 gram. In some embodiments, the dosage of the composition administered about 0.5 grams, about 0.6 grams, about 0.7 grams, about 0.8 grams, about 0.9 grams, or about 1 gram. In some embodiments, the dosage of the composition administered at least about 0.5 grams, about 0.6 grams, about 0.7 grams, about 0.8 grams, or about 0.9 grams. In some embodiments, the dosage of the composition administered at most about 0.6 grams, about 0.7 grams, about 0.8 grams, about 0.9 grams, or about 1 gram. In some embodiments, the dosage of the composition administered about 1 gram to about 2 grams. In some embodiments, the dosage of the composition administered about 1 gram to about 1.1 grams, about 1 gram to about 1.2 grams, about 1 gram to about 1.3 grams, about 1 gram to about 1.4 grams, about 1 gram to about 1.5 grams, about 1 gram to about 1.6 grams, about 1 gram to about 1.7 grams, about 1 gram to about 1.8 grams, about 1 gram to about 1.9 grams, about 1 gram to about 2 grams, about 1.1 grams to about 1.2 grams, about 1.1 grams to about 1.3 grams, about 1.1 grams to about 1.4 grams, about 1.1 grams to about 1.5 grams, about 1.1 grams to about 1.6 grams, about 1.1 grams to about 1.7 grams, about 1.1 grams to about 1.8 grams, about 1.1 grams to about 1.9 grams, about 1.1 grams to about 2 grams, about 1.2 grams to about 1.3 grams, about 1.2 grams to about 1.4 grams, about 1.2 grams to about 1.5 grams, about 1.2 grams to about 1.6 grams, about 1.2 grams to about 1.7 grams, about 1.2 grams to about 1.8 grams, about 1.2 grams to about 1.9 grams, about 1.2 grams to about 2 grams, about 1.3 grams to about 1.4 grams, about 1.3 grams to about 1.5 grams, about 1.3 grams to about 1.6 grams, about 1.3 grams to about 1.7 grams, about 1.3 grams to about 1.8 grams, about 1.3 grams to about 1.9 grams, about 1.3 grams to about 2 grams, about 1.4 grams to about 1.5 grams, about 1.4 grams to about 1.6 grams, about 1.4 grams to about 1.7 grams, about 1.4 grams to about 1.8 grams, about 1.4 grams to about 1.9 grams, about 1.4 grams to about 2 grams, about 1.5 grams to about 1.6 grams, about 1.5 grams to about 1.7 grams, about 1.5 grams to about 1.8 grams, about 1.5 grams to about 1.9 grams, about 1.5 grams to about 2 grams, about 1.6 grams to about 1.7 grams, about 1.6 grams to about 1.8 grams, about 1.6 grams to about 1.9 grams, about 1.6 grams to about 2 grams, about 1.7 grams to about 1.8 grams, about 1.7 grams to about 1.9 grams, about 1.7 grams to about 2 grams, about 1.8 grams to about 1.9 grams, about 1.8 grams to about 2 grams, or about 1.9 grams to about 2 grams. In some embodiments, the dosage of the composition administered about 1 gram, about 1.1 grams, about 1.2 grams, about 1.3 grams, about 1.4 grams, about 1.5 grams, about 1.6 grams, about 1.7 grams, about 1.8 grams, about 1.9 grams, or about 2 grams. In some embodiments, the dosage of the composition administered at least about 1 gram, about 1.1 grams, about 1.2 grams, about 1.3 grams, about 1.4 grams, about 1.5 grams, about 1.6 grams, about 1.7 grams, about 1.8 grams, or about 1.9 grams. In some embodiments, the dosage of the composition administered at most about 1.1 grams, about 1.2 grams, about 1.3 grams, about 1.4 grams, about 1.5 grams, about 1.6 grams, about 1.7 grams, about 1.8 grams, about 1.9 grams, or about 2 grams. In some embodiments, the dosage of the composition administered about 2 grams to about 5 grams. In some embodiments, the dosage of the composition administered about 2 grams to about 2.25 grams, about 2 grams to about 2.5 grams, about 2 grams to about 2.75 grams, about 2 grams to about 3 grams, about 2 grams to about 3.5 grams, about 2 grams to about 4 grams, about 2 grams to about 4.5 grams, about 2 grams to about 5 grams, about 2.25 grams to about 2.5 grams, about 2.25 grams to about 2.75 grams, about 2.25 grams to about 3 grams, about 2.25 grams to about 3.5 grams, about 2.25 grams to about 4 grams, about 2.25 grams to about 4.5 grams, about 2.25 grams to about 5 grams, about 2.5 grams to about 2.75 grams, about 2.5 grams to about 3 grams, about 2.5 grams to about 3.5 grams, about 2.5 grams to about 4 grams, about 2.5 grams to about 4.5 grams, about 2.5 grams to about 5 grams, about 2.75 grams to about 3 grams, about 2.75 grams to about 3.5 grams, about 2.75 grams to about 4 grams, about 2.75 grams to about 4.5 grams, about 2.75 grams to about 5 grams, about 3 grams to about 3.5 grams, about 3 grams to about 4 grams, about 3 grams to about 4.5 grams, about 3 grams to about 5 grams, about 3.5 grams to about 4 grams, about 3.5 grams to about 4.5 grams, about 3.5 grams to about 5 grams, about 4 grams to about 4.5 grams, about 4 grams to about 5 grams, or about 4.5 grams to about 5 grams. In some embodiments, the dosage of the composition administered about 2 grams, about 2.25 grams, about 2.5 grams, about 2.75 grams, about 3 grams, about 3.5 grams, about 4 grams, about 4.5 grams, or about 5 grams. In some embodiments, the dosage of the composition administered at least about 2 grams, about 2.25 grams, about 2.5 grams, about 2.75 grams, about 3 grams, about 3.5 grams, about 4 grams, or about 4.5 grams. In some embodiments, the dosage of the composition administered at most about 2.25 grams, about 2.5 grams, about 2.75 grams, about 3 grams, about 3.5 grams, about 4 grams, about 4.5 grams, or about 5 grams. In some embodiments, the dosage of the composition administered about 5 grams to about 10 grams. In some embodiments, the dosage of the composition administered about 5 grams to about 6 grams, about 5 grams to about 7 grams, about 5 grams to about 8 grams, about 5 grams to about 9 grams, about 5 grams to about 10 grams, about 6 grams to about 7 grams, about 6 grams to about 8 grams, about 6 grams to about 9 grams, about 6 grams to about 10 grams, about 7 grams to about 8 grams, about 7 grams to about 9 grams, about 7 grams to about 10 grams, about 8 grams to about 9 grams, about 8 grams to about 10 grams, or about 9 grams to about 10 grams. In some embodiments, the dosage of the composition administered about 5 grams, about 6 grams, about 7 grams, about 8 grams, about 9 grams, or about 10 grams. In some embodiments, the dosage of the composition administered at least about 5 grams, about 6 grams, about 7 grams, about 8 grams, or about 9 grams. In some embodiments, the dosage of the composition administered at most about 6 grams, about 7 grams, about 8 grams, about 9 grams, or about 10 grams. In some embodiments, the dosage of the composition administered about 10 grams to about 32 grams. In some embodiments, the dosage of the composition administered about 10 grams to about 12.5 grams, about 10 grams to about 15 grams, about 10 grams to about 17.5 grams, about 10 grams to about 20 grams, about 10 grams to about 24 grams, about 10 grams to about 28 grams, about 10 grams to about 32 grams, about 12.5 grams to about 15 grams, about 12.5 grams to about 17.5 grams, about 12.5 grams to about 20 grams, about 12.5 grams to about 24 grams, about 12.5 grams to about 28 grams, about 12.5 grams to about 32 grams, about 15 grams to about 17.5 grams, about 15 grams to about 20 grams, about 15 grams to about 24 grams, about 15 grams to about 28 grams, about 15 grams to about 32 grams, about 17.5 grams to about 20 grams, about 17.5 grams to about 24 grams, about 17.5 grams to about 28 grams, about 17.5 grams to about 32 grams, about 20 grams to about 24 grams, about 20 grams to about 28 grams, about 20 grams to about 32 grams, about 24 grams to about 28 grams, about 24 grams to about 32 grams, or about 28 grams to about 32 grams. In some embodiments, the dosage of the composition administered about 10 grams, about 12.5 grams, about 15 grams, about 17.5 grams, about 20 grams, about 24 grams, about 28 grams, or about 32 grams. In some embodiments, the dosage of the composition administered at least about 10 grams, about 12.5 grams, about 15 grams, about 17.5 grams, about 20 grams, about 24 grams, or about 28 grams. In some embodiments, the dosage of the composition administered at most about 12.5 grams, about 15 grams, about 17.5 grams, about 20 grams, about 24 grams, about 28 grams, or about 32 grams.

In some embodiments, the composition may be applied to the skin region at a frequency. The frequency may be about once daily, twice daily, three times daily, four times daily, five times daily, six times daily, about once every two days, about once every three days, about once every four days, about once every five days, about once every six days, about once every seven days, about twice every three days, about twice every five days, or about twice every seven days. While certain frequencies are listed, these are exemplary and the composition may be applied at other frequencies. Additionally, the composition may be applied whenever symptoms arise. The composition may be applied at the frequency for a duration. The duration may be one day, two days, three days, four days, five days, six days, seven days, about one week, about two weeks, about three weeks, about four weeks, about five weeks, about six weeks, about one month, about two months, about three months, about four months, about five months, about six months, about seven months, about eight months, about nine months, about ten months, about eleven months, about twelve months, about one year, about two years, about three years, about four years, about five years, about six years, about seven years, about eight years, about nine years, or about ten years. While certain durations are listed, these are exemplary and the frequency at which the composition is applied may last for alternate durations.

### Exemplary Embodiments

Among the exemplary embodiments are:
1. A composition, comprising:
   an internal phase in an external phase,
   wherein the internal phase, the external phase, or both comprise:
      a prebiotic,
      an antioxidant mixture, and
      a moisturizer mixture, and
   wherein the internal phase and the external phase are a reverse emulsion.
2. The composition of embodiment 1, wherein the composition is formulated for external administration to a skin region of a mammal.
3. The composition of embodiment 1 or 2, wherein the prebiotic comprises xylitol, lactitol, a fructan, a polysaccharide, an oligosaccharide, levan, polymnia sonchifolia root juice, or any combination of two or more thereof.
4. The composition of embodiment 3, wherein the fructan, the polysaccharide, and/or oligosaccharide comprise pectin, beta-glucan, inulin, bombax costatum flower extract, or any combination of two or more thereof.
5. The composition of any one of embodiments 1 to 4, wherein the antioxidant mixture comprises tetrahexyldecyl ascorbate, ubiquinone, resveratrol, epigallocatechin gallate, ellagic acid, ursolic acid, oleanolic acid, chlorogenic acid, rutin, hesperidin, quercetin, bakuchiol, botanical extracts of the leaves, flowers, seeds, fruits, stems, barks, or roots of a plant, vitamins, or any combination of two or more thereof.
6. The composition of embodiment 5, wherein,
   the botanical extracts of the leaves, flowers, seeds, fruits, stems, barks, or roots of the plant comprise Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract, or any combination of two or more thereof, and
   the vitamins or derivatives thereof comprise tocopherol, tocopheryl acetate, ubiquinone, ascorbic acid, magnesium ascorbyl phosphate, tetrahexyldecyl ascorbate, retinol, retinaldehyde, retinyl palmitate, or any combination of two or more thereof.
7. The composition of embodiment 6, wherein the antioxidant mixture comprises tocopherol.
8. The composition of any one of embodiments 1 to 7, wherein the moisturizer mixture comprises dimethicone, shea butter, jojoba esters, Helianthus annuus (sunflower) seed wax; Helianthus annuus (sunflower) seed oil, Simmondsia chinensis (jojoba) seed oil, Olea europaea (olive) oil, Glycine soja (soybean) oil, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, cetyl esters, or any combination of two or more thereof.
9. The composition of any one of embodiments 1 to 8, wherein the internal phase, external phase, or both comprises dipalmitoyl hydroxyproline.
10. The composition of any one of embodiments 1 to 9, wherein the internal phase, external phase, or both further comprise a carbohydrate or derivative thereof.
11. The composition of embodiment 10, wherein the carbohydrate comprises biosaccharide gum-1.
12. The composition of any one of embodiments 1 to 11, wherein the internal phase, external phase, or both further comprise one or more excipients.
13. The composition of embodiment 12, wherein the one or more excipients comprises caprylic/capric triglyceride, glycerin, squalane, dimethicone, glyceryl behenate/eicosadioate, magnesium sulfate, steareth-2, oleth-2, oleth-7, ceteareth-20, sorbitan oleate, sorbitan stearate, sorbitan isostearate, sorbitan laurate, sorbitan olivate, glyceryl oleate, glyceryl stearate, glyceryl ricinoleate, glyceryl caprylate, glyceryl undecylenate, sucrose distearate, peg-20 methyl glucose sesquistearate, peg-30 polyhydroxystearate, polyglyceryl-2 sesquiisostearate, polyglyceryl-2 sesquioleate, palmitic acid, polyglyceryl-2 sesquicaprylate, polyglyceryl-2 diisostearate, polyglyceryl-2 dipolyhydroxystearate, polyglyceryl-2 oleate, polyglyceryl-2 stearate, polyglyceryl-3 oleate, polyglyceryl-3 behenate, polyglyceryl-3 diisostearate, polyglyceryl-3 triolivate, polyglyceryl-3 ricinolate, diisostearoyl polyglyceryl-3 dimer dilinolate, polyglyceryl-3 polyricinoleate, polyglyceryl-3 dimer dilinoleate, polyglyceryl-3 sorbityl linseedate, polyglyceryl-3 polydimethylsiloxyethyl dimethicone, polyglyceryl-3 methylglucose dioleate, polyglyceryl-4 stearate, polyglyceryl-4 oleate, polyglyceryl-4 isostearate, polyglyceryl-4 diisostearate/polyhydroxy stearate/sebacate, polyglyceryl-4 olivate/polyricinolate, polyglyceryl-6 oleate, polyglyceryl-6 pentaoleate, olive oil polyglyceryl-6 esters, polyglyceryl-6 polyricinolate, polyglyceryl-6 polyhydroxystearate, polyglyceryl-10 hexaoleate, polyglyceryl-10 diolate, polyglyceryl-10 stearate, methyl glucose sesquistearate, methyl glucose dioleate hydrogenated lecithin, cetyl peg/ppg-10/1 dimethicone, peg-10 dimethicone, peg/ppg-20/15 dimethicone, peg/ppg-18/18 dimethicone, ppg-8 dimethicone, peg-9 methyl ether dimethicone, bis-peg/ppg-14/14 dimethicone, dimethicone/peg-10/15 crosspolymer, lauryl peg-9 polydimethylsiloxethyl dimethicone, lauryl dimethicone/polyglycerin-3 crosspolymer, lauryl peg-8 dimethicone, lauryl polyglyceryl-3 polydimethylsiloxethyl dimethicone, cetyl diglyceryl tris(trimethylsiloxy)silyethyl dimethicone, sodium hydroxide, ammonium hydroxide, triethanolamine, aminomethyl propanol, citric acid, phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, tetrasodium glutamate diacetate, disodium EDTA, tetrasodium edta, etidronic acid, trisodium dicarboxymethyl alaninate, trisodium ethylenediamine disuccinate, sodium phytate, or any combination of two or more thereof.
14. The composition of any one of embodiments 1 to 13, wherein the skin region is vulvar, penile, or scrotal.
15. The composition of any one of embodiments 2 to 14, wherein the mammal is a human.
16. The composition of embodiment 15, wherein the human is female.
17. The composition of any one of embodiments 14 to 16, wherein the skin region is vulvar.
18. The composition of embodiment 15, wherein the human is male.
19. The composition of any one of embodiments 14, 15, or 18, wherein the skin region is penile or scrotal.
20. The composition of any one of embodiments 1 to 19, wherein a level of moisture in the skin region is increased relative to a reference level of moisture of the skin region after externally administering the composition.
21. The composition of any one of embodiments 1 to 20, wherein a redness of the skin region is improved relative to a reference redness of the skin region after externally administering the composition.
22. The composition of any one of embodiments 1 to 21, wherein the composition is a formulation.
23. The composition of embodiment 22, wherein the formulation is a cream, lotion, serum, or solid-stick.
24. The composition of any one of embodiments 1 to 23, wherein the composition is formulated for topical administration to the skin region.
25. The composition of any one of embodiments 15 to 24, wherein the human experiencing menopausal symptoms, perimenopausal symptoms, premenopausal symptoms, or postmenopausal symptoms.
26. The composition of embodiment 25, wherein the menopausal symptoms, perimenopausal symptoms, premenopausal symptoms, or postmenopausal symptoms comprise dryness, irritation, itch, or redness of the skin region.
27. The composition of embodiment 25 or 26, wherein the menopausal symptoms, perimenopausal symptoms, premenopausal symptoms, or postmenopausal symptoms are reduced after administering the composition.
28. The composition of any one of embodiments 15 to 27, wherein the human is premenopausal.
29. The composition of any one of embodiments 1 to 28, wherein the composition is formulated to be administered at a dosage of about 0.01 grams to 10 grams.
30. The composition of any one of embodiments 1 to 29, wherein the prebiotic is present in the composition at about 0.001% to about 5%
31. The composition of any one of embodiments 1 to 30, wherein the prebiotic is present in the composition at about 3% to about 5%.
32. The composition of any one of embodiments 1 to 31, wherein the antioxidant mixture is present in the composition at about to about 0.001% to 5%.
33. The composition of any one of embodiments 1 to 32, wherein the antioxidant mixture is present in the composition at about 3% to about 5%.
34. The composition of any one of embodiments 1 to 33, wherein the moisturizer mixture is present in the composition at about 0.001% to about 80%.
35. The composition of any one of embodiments 1 to 34, wherein the moisturizer mixture is present in the composition at about 40% to about 80%.
36. The composition of any one of embodiments 1 to 35, wherein the internal phase comprises a hydrator or humectant.
37. The composition of embodiment 36, wherein the hydrator or humectant comprises hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10, or any combination of two or more thereof.
38. The composition of any one of embodiments 1 to 37, wherein the internal phase is a water phase.
39. The composition of any one of embodiments 1 to 38, wherein the external phase is an oil phase or a silicone phase.
40. The composition of embodiment 1 to 39, wherein the internal phase, external phase, or both comprise the antioxidant mixture.
41. The composition of embodiment 40, wherein the antioxidant mixture comprises Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract, or any combination of two or more thereof.
42. The composition of any one of embodiments 1 to 41, wherein a level of smoothness of the skin region is improved relative to a reference level of smoothness of the skin region after externally administering the composition.
43. The composition of any one of embodiments 1 to 42, wherein a level of irritation of the skin region is improved relative to a reference level of irritation of the skin region after externally administering the composition.
44. The composition of any one of embodiments 1 to 43, wherein a texture of the skin region is improved relative to a reference texture of the skin region after externally administering the composition.
45. The composition of any one of embodiments 1 to 44, wherein the skin region is soothed after externally administering the composition.
46. The composition of any one of embodiments 1 to 45, wherein the composition is externally administered after the skin region is shaved.
47. The composition of any one of embodiments 1 to 46, wherein the internal phase comprises the antioxidant mixture.
48. The composition of any one of embodiments 1 to 46, wherein the external phase comprises the antioxidant mixture.
49. The composition of any one of embodiments 1 to 48, wherein the internal phase comprises the moisturizer mixture.
50. The composition of any one of embodiments 1 to 48, wherein the external phase comprises the moisturizer mixture.
51. The composition of any one of embodiments 1 to 50, wherein the internal phase comprises the carbohydrate.
52. The composition of any one of embodiments 1 to 50, wherein the external phase comprises the carbohydrate.
53. The composition of any one of embodiments 1 to 46, wherein:
   a. the internal phase comprises the antioxidant mixture; and
   b. the external phase comprises the moisturizer mixture.
54. The composition of embodiment 53, wherein the external phase comprises the carbohydrate.
55. A method for increasing moisture in a skin region of a mammal, comprising externally administering a composition formulated as a reverse emulsion to the skin region, the composition comprising:
   an internal phase in an external phase, wherein the internal phase, external phase, or both comprise:
      a prebiotic;
      an antioxidant mixture; and
      a moisturizer mixture.
56. The method of embodiment 55, wherein the prebiotic comprises xylitol, lactitol, a fructan, a polysaccharide, an oligosaccharide, levan, polymnia sonchifolia root juice, or any combination of two or more thereof.
57. The method of embodiment 56, wherein the fructan, the polysaccharide, and/or oligosaccharide comprise Pectin, Beta-Glucan, Inulin, Bombax Costatum Flower Extract, or any combination of two or more thereof.
58. The method of any one of embodiments 55 to 57, wherein the antioxidant mixture comprises tetrahexyldecyl ascorbate, ubiquinone, resveratrol, epigallocatechin gallate, ellagic acid, ursolic acid, oleanolic acid, chlorogenic acid, rutin, hesperidin, quercetin, bakuchiol, botanical extracts of the leaves, flowers, seeds, fruits, stems, barks, or roots of a plant, vitamins or derivatives thereof, or any combination of two or more thereof.
59. The method of embodiment 58, wherein,
   the botanical extracts of the leaves, flowers, seeds, fruits, stems, barks, or roots of the plant comprise Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract, or any combination of two or more thereof, and
   the vitamins or derivatives thereof comprise tocopherol, tocoopheryl acetate, ubiquinone, ascorbic acid, magnesium ascorbyl phosphate, tetrahexyldecyl ascorbate, retinol, retinaldehyde, retinyl palmitate, or any combination of two or more thereof.
60. The method of any one of embodiments 55 to 59, wherein the antioxidant mixture comprises tocopherol.
61. The method of any one of embodiments 55 to 60, wherein the moisturizer mixture comprises dimethicone, shea butter, jojoba esters, Helianthus annuus (sunflower) seed wax; Helianthus annuus (sunflower) seed oil, Simmondsia chinensis (jojoba) seed oil, Olea europaea (olive) oil, Glycine soja (soybean) oil, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, cetyl esters, or any combination of two or more thereof.
62. The method of any one of embodiments 55 to 61, wherein the internal phase, external phase, or both further comprise dipalmitoyl hydroxyproline.
63. The method of any one of embodiments 55 to 62, wherein the internal phase, external phase, or both comprises a carbohydrate or derivative thereof.
64. The method of embodiment 63, wherein the carbohydrate comprises biosaccharide gum-1.
65. The method of any one of embodiments 55 to 64, wherein the internal phase, external phase, or both further comprise one or more excipients.
66. The method of embodiment 65, wherein the one or more excipients comprises caprylic/capric triglyceride, glycerin, squalane, dimethicone, glyceryl behenate/eicosadioate, magnesium sulfate, steareth-2, oleth-2, oleth-7, ceteareth-20, sorbitan oleate, sorbitan stearate, sorbitan isostearate, sorbitan laurate, sorbitan olivate, glyceryl oleate, glyceryl stearate, glyceryl ricinoleate, glyceryl caprylate, glyceryl undecylenate, sucrose distearate, peg-20 methyl glucose sesquistearate, peg-30 polyhydroxystearate, polyglyceryl-2 sesquiisostearate, polyglyceryl-2 sesquioleate, palmitic acid, polyglyceryl-2 sesquicaprylate, polyglyceryl-2 diisostearate, polyglyceryl-2 dipolyhydroxystearate, polyglyceryl-2 oleate, polyglyceryl-2 stearate, polyglyceryl-3 oleate, polyglyceryl-3 behenate, polyglyceryl-3 diisostearate, polyglyceryl-3 triolivate, polyglyceryl-3 ricinolate, diisostearoyl polyglyceryl-3 dimer dilinolate, polyglyceryl-3 polyricinoleate, polyglyceryl-3 dimer dilinoleate, polyglyceryl-3 sorbityl linseedate, polyglyceryl-3 polydimethylsiloxyethyl dimethicone, polyglyceryl-3 methylglucose dioleate, polyglyceryl-4 stearate, polyglyceryl-4 oleate, polyglyceryl-4 isostearate, polyglyceryl-4 diisostearate/polyhydroxy stearate/sebacate, polyglyceryl-4 olivate/polyricinolate, polyglyceryl-6 oleate, polyglyceryl-6 pentaoleate, olive oil polyglyceryl-6 esters, polyglyceryl-6 polyricinolate, polyglyceryl-6 polyhydroxystearate, polyglyceryl-10 hexaoleate, polyglyceryl-10 diolate, polyglyceryl-10 stearate, methyl glucose sesquistearate, methyl glucose dioleate hydrogenated lecithin, cetyl peg/ppg-10/1 dimethicone, peg-10 dimethicone, peg/ppg-20/15 dimethicone, peg/ppg-18/18 dimethicone, ppg-8 dimethicone, peg-9 methyl ether dimethicone, bis-peg/ppg-14/14 dimethicone, dimethicone/peg-10/15 crosspolymer, lauryl peg-9 polydimethylsiloxethyl dimethicone, lauryl dimethicone/polyglycerin-3 crosspolymer, lauryl peg-8 dimethicone, lauryl polyglyceryl-3 polydimethylsiloxethyl dimethicone, cetyl diglyceryl tris (trimethylsiloxy)silyethyl dimethicone, sodium hydroxide, ammonium hydroxide, triethanolamine, aminomethyl propanol, citric acid, phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, tetrasodium glutamate diacetate, disodium EDTA, tetrasodium edta, etidronic acid, trisodium dicarboxymethyl alaninate, trisodium ethylenediamine disuccinate, sodium phytate, or any combination of two or more thereof.
67. The method of any one of embodiments 55 to 66, wherein the skin region is vulvar, penile, or scrotal.
68. The method of any one of embodiments 56 to 67, wherein the mammal is a human.
69. The method of embodiment 68, wherein the human is female.
70. The method of any one of embodiments 67 to 69, wherein the skin region is vulvar.
71. The method of embodiment 68, wherein the human is male.
72. The method of any one of embodiments 67, 68, or 71, wherein the skin region is penile or scrotal.
73. The method of any one of embodiments 55 to 72, wherein administering the composition to the skin region comprises administering the composition to the skin region at a frequency of once daily, twice daily, thrice daily, four times daily, five times daily, or six times daily.
74. The method of embodiment 73, wherein the frequency is twice daily.
75. The method of any one of embodiments 55 to 74, wherein a level of moisture in the skin region is increased relative to a reference level of moisture in the skin region after externally administering the composition.
76. The method of any one of embodiments 55 to 75, wherein a redness of the skin region is improved relative to a reference redness of the skin region after externally administering the composition.
77. The method of any one of embodiments 55 to 76, wherein the composition is a formulation.
78. The method of embodiment 77, wherein the formulation is a cream, serum, lotion, or solid-stick.
79. The method of any one of embodiments 55 to 78, wherein externally administering the composition to the skin region comprises topically applying the composition to the skin region.
80. The method of any one of embodiments 68 to 79, wherein the human is experiencing menopausal symptoms, perimenopausal symptoms, premenopausal symptoms, or postmenopausal symptoms.
81. The method of embodiment 80, wherein the menopausal symptoms, perimenopausal symptoms, premenopausal symptoms, or postmenopausal symptoms comprise dryness, irritation, itch, or redness of the skin region.
82. The method of embodiment 55 or 81, wherein the menopausal symptoms, perimenopausal symptoms, premenopausal symptoms, or postmenopausal symptoms are reduced after administering the composition.
83. The method of embodiment 68, wherein the human is premenopausal.
84. The method of any one of embodiments 55 to 83, wherein externally administering the composition to the skin region comprises administering the composition at a dosage of about 0.1 grams to about 10 grams.
85. The method of any one of embodiments 55 to 84, wherein the prebiotic is present in the composition to about 0.001% to about 5%
86. The method of any one of embodiments 55 to 85, wherein the prebiotic is present in the composition at about 3% to about 5%.
87. The method of any one of embodiments 55 to 86, wherein the antioxidant mixture is present in the composition at about 0.001% to about 5%.
88. The method of any one of embodiments 55 to 87, wherein the antioxidant mixture is present in the composition at about 3% to about 5%.
89. The method of any one of embodiments 55 to 88, wherein the moisturizer mixture is present in the composition to about 0.001% to about 80%.
90. The method of any one of embodiments 55 to 89, wherein the moisturizer mixture is present in the composition at about 40% to about 80%.
91. The method of any one of embodiments 55 to 90, wherein externally administering the composition results in increased collagen production in the skin region.
92. The method of any one of embodiments 55 to 91, wherein the internal phase comprises a hydrator or humectant.
93. The method of embodiment 92, wherein the hydrator or humectant comprises hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10, or any combination of two or more thereof.
94. The method of any one of embodiments 55 to 93, wherein the internal phase is a water phase.
95. The method of any one of embodiments 55 to 94, wherein the external phase is an oil phase or a silicone phase.
96. The method of embodiment 55 to 95, wherein the internal phase, external phase, or both comprises the antioxidant mixture.
97. The method of embodiment 96, wherein the antioxidant mixture comprises Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract, or any combination of two or more thereof.
98. The method of any one of embodiments 55 to 97, wherein a level of smoothness of the skin region is improved relative to a reference level of smoothness of the skin region after externally administering the composition.
99. The method of any one of embodiments 55 to 98, wherein a level of irritation of the skin region is improved relative to a reference level of irritation of the skin region after externally administering the composition.
100. The method of any one of embodiments 55 to 99, wherein a texture of the skin region is improved relative to a reference texture of the skin region after externally administering the composition.
101. The method of any one of embodiments 55 to 100, wherein the skin region is soothed after externally administering the composition.
102. The method of any one of embodiments 55 to 101, wherein the composition is externally administered after the skin region is shaved.
103. The method of any one of embodiments 55 to 102, wherein the internal phase comprises the antioxidant mixture.
104. The method of any one of embodiments 55 to 102, wherein the external phase comprises the antioxidant mixture.
105. The method of any one of embodiments 55 to 104, wherein the internal phase comprises the moisturizer mixture.
106. The method of any one of embodiments 55 to 104, wherein the external phase comprises the moisturizer mixture.
107. The method of any one of embodiments 55 to 106, wherein the internal phase comprises the carbohydrate.
108. The method of any one of embodiments 55 to 106, wherein the external phase comprises the carbohydrate.
109. The method of any one of embodiments 55 to 108, wherein:
   a. the internal phase comprises the antioxidant mixture; and
   b. the external phase comprises the moisturizer mixture.
110. The method of embodiment 109, wherein the external phase comprises the carbohydrate.

### EXAMPLES

The following illustrative examples are representative of embodiments of the compositions and methods described herein and are not meant to be limiting in any way.

### Example 1—Formulation for Application to Vulva

A lotion formulated as a reverse emulsion comprising a water phase in an oil phase is applied to the vulva of a human female. The lotion is 20% water phase by weight and 80% oil phase by weight. The water phase includes the prebiotics xylitol, lactitol, and Beta-Glucan which together make up about 5% of the lotion by weight. The water phase further includes hyaluronic acid and butylene glycol as hydrators, which makes up about 5% of the lotion by weight. The oil phase includes an antioxidant mixture, which makes up about 4% of the lotion by weight. The antioxidant mixture includes tetrahexyldecyl ascorbate, ubiquinone, oleanolic acid, and Camellia sinensis (green tea) extract. The oil phase further includes a moisturizer mixture, which makes up about 40% of the lotion by weight. The moisturizer mixture includes jojoba esters, shea butter, dimethicone, and squalane. The oil phase additionally includes dipalmitoyl hydroxyproline, which makes up about 2% of the lotion by weight. The lotion additionally includes biosaccharide gum-1, which makes up about 0.5-1% of the lotion by weight. The lotion further includes one or more excipients including glyceryl eicosadioate, polyglyceryl-6 polyricinoleate as emulsifiers, sodium hydroxide as a pH adjustor, phenoxyethanol as a preservative, and disodium EDTA as a chelating agent, where the one or more excipients make up the remainder of the lotion by weight.

After the lotion is applied to the vulva, redness of the vulva is reduced as well as the dryness of the vulva is reduced.

### Example 2—Formulation for Application to Vulva

A cream formulated as a reverse emulsion comprising a water phase in an oil phase is applied to the vulva of a human female. The cream is 30% water phase by weight and 70% oil phase by weight. The water phase includes the prebiotics xylitol and inulin which together make up about 5% of the cream by weight. The water phase further includes Bacillus Ferment Extract as a antioxidant mixture, which makes up about 3% of the cream by weight. The water phase further includes saccharide isomerate as a hydrator, which makes up about 3% to 4% of the cream by weight. The oil phase includes an antioxidant mixture, which makes up about 2% of the cream by weight. The antioxidant mixture includes tetrahexyldecyl ascorbate, ubiquinone, and chlorogenic acid. The oil phase further includes a moisturizer mixture, which makes up about 35% of the cream by weight. The moisturizer mixture includes jojoba esters, Theobroma cacao (cocoa) seed butter, and dimethicone. The oil phase additionally includes dipalmitoyl hydroxyproline, which makes up about 2% of the cream by weight. The cream additionally includes biosaccharide gum-1, which makes up about 4% of the cream by weight. The cream further includes one or more excipients including glyceryl eicosadioate, polyglyceryl-6 polyricinoleate as emulsifiers, ammonium hydroxide as a pH adjustor, phenoxyethanol as a preservative, and tetrasodium EDTA as a chelating agent, where the one or more excipients make up the remainder of the cream by weight.

After the cream is applied to the vulva, redness of the vulva is reduced as well as the dryness of the vulva is reduced.

### Example 3—Formulation for Application to Vulva

A cream formulated as a reverse emulsion comprising a water phase in an oil phase is applied to the vulva of a human female. The cream is 55% water phase by weight and 45% oil phase by weight. The water phase includes the prebiotics xylitol and lactitol, which together make up about 3% of the cream by weight. The water phase further includes hyaluronic acid as a hydrator, which makes up about 0.2% of the cream by weight. The oil phase includes an antioxidant mixture, which makes up about 0.5% of the cream by weight. The antioxidant mixture includes tetrahexyldecyl ascorbate, ubiquinone, and vitamin E. The oil phase further includes a moisturizer mixture, which makes up about 28% of the cream by weight. The moisturizer mixture includes jojoba esters, shea butter, squalane, and dimethicone. The oil phase additionally includes dipalmitoyl hydroxyproline, which makes up about 1% of the cream by weight. The cream additionally includes biosaccharide gum-1, which makes up about 5% of the cream by weight. The cream further includes one or more excipients including polyglyceryl-6 polyhydroxystearate as an emulsifier, sodium hydroxide as a pH adjustor, phenoxyethanol as a preservative, and disodium EDTA as a chelating agent, where the one or more excipients make up the remainder of the cream by weight.

After the cream is applied to the vulva, redness of the vulva is reduced as well as the dryness of the vulva is reduced.

### Example 4—Formulation for Application to Penis and Scrotum

A cream formulated as a reverse emulsion comprising a water phase in an oil phase is applied to the penis and scrotum of a human male. The cream is 50% water phase by weight and 50% oil phase by weight. The water phase includes the prebiotics xylitol and lactitol, which together make up about 3% of the cream by weight. The water phase further includes hyaluronic acid as a hydrator, which makes up about 0.3% to 0.5% of the cream by weight. The oil phase includes an antioxidant mixture, which makes up about 5% of the cream by weight. The antioxidant mixture includes tetrahexyldecyl ascorbate, ubiquinone, and vitamin E. The oil phase further includes a moisturizer mixture, which makes up about 15% of the cream by weight. The moisturizer mixture includes jojoba esters, shea butter, and dimethicone. The oil phase additionally includes dipalmitoyl hydroxyproline, which makes up about 3% of the cream by weight. The cream additionally includes biosaccharide gum-1, which makes up about 5% of the cream by weight. The cream further includes one or more excipients including polyglyceryl-6 polyhydroxystearate as an emulsifier, triethanolamine as a pH adjustor, sorbic acid as a preservative, and sodium phytate as a chelating agent, where the one or more excipients make up the remainder of the cream by weight.

After the cream is applied to the penis and scrotum, redness of the penis and scrotum is reduced as well as the dryness of the penis and scrotum is reduced.

### Example 5—Formulation for Application to Penis and Scrotum

A serum formulated as a reverse emulsion comprising a water phase in an oil phase is applied to the penis and scrotum of a human male. The serum is 40% water phase by weight and 60% oil phase by weight. The water phase includes the prebiotic xylitol, which together make up about 3% of the serum by weight. The water phase further includes hyaluronic acid and honey as a hydrator and humectant, respectively, which make up about 1% of the serum by weight. The oil phase includes an antioxidant mixture, which makes up about 5% of the serum by weight. The antioxidant mixture includes tetrahexyldecyl ascorbate, ubiquinone, Punica granatum (pomegranate) extract, and vitamin E. The oil phase further includes a moisturizer mixture, which makes up about 30% of the serum by weight. The moisturizer mixture includes shea butter, and dimethicone. The oil phase additionally includes dipalmitoyl hydroxyproline, which makes up about 3% of the serum by weight. The serum additionally includes biosaccharide gum-1, which makes up about 3% of the serum by weight. The serum further includes one or more excipients including methyl glucose dioleate hydrogenated lecithin as an emulsifier, aminomethyl propanol as a pH adjustor, methylparaben as a preservative, and trisodium ethylenediamine disuccinate as a chelating agent, where the one or more excipients make up the remainder of the serum by weight.

After the serum is applied to the penis and scrotum, redness of the penis and scrotum is reduced as well as the dryness of the penis and scrotum is reduced.

### Example 6—Formulation for Application to Vulva

A cream formulated as a reverse emulsion comprising a water phase in an oil phase is applied to the vulva of a human female. The cream is 55% water phase by weight and 45% oil phase by weight. The water phase includes the prebiotics xylitol and lactitol, which together make up about 3% of the cream by weight. The water phase further includes hyaluronic acid as a hydrator, which makes up about 0.2% of the cream by weight. The water phase further includes the antioxidant mixture bacillus ferment extract, which makes up about 3% of the cream by weight. The oil phase includes an antioxidant mixture, which makes up about 1% of the cream by weight. The antioxidant mixture includes tetrahexyldecyl ascorbate, ubiquinone, and vitamin E. The oil phase further includes a moisturizer mixture, which makes up about 25% of the cream by weight. The moisturizer mixture includes jojoba esters, shea butter, squalane, and dimethicone. The oil phase additionally includes dipalmitoyl hydroxyproline, which makes up about 1% of the cream by weight. The cream additionally includes biosaccharide gum-1, which makes up about 5% of the cream by weight. The cream further includes one or more excipients including polyglyceryl-6 polyhydroxystearate as an emulsifier, sodium hydroxide as a pH adjustor, phenoxyethanol as a preservative, and disodium EDTA as a chelating agent, where the one or more excipients make up the remainder of the cream by weight.

After the cream is applied to the vulva, redness of the vulva is reduced as well as the dryness of the vulva is reduced.

### Example 7—Application of Formulation to Vulva After Shaving

A cream formulated as a reverse emulsion comprising a water phase in an oil phase was applied to the vulva of 25 human females between the ages of 23 years old and 61 years old. The cream is 50% water phase by weight and 50% oil phase by weight, where the oil phase was the external phase. The water phase includes the prebiotics xylitol and lactitol, which together make up about 3% of the cream by weight. The water phase further includes hyaluronic acid as a hydrator, which makes up about 10% of the cream by weight. The oil phase includes an antioxidant mixture, which makes up about 5% of the cream by weight. The antioxidant mixture includes tetrahexyldecyl ascorbate, ubiquinone, and vitamin E. The oil phase further includes a moisturizer mixture, which makes up about 10% of the cream by weight. The moisturizer mixture includes jojoba esters, shea butter, and dimethicone. The oil phase additionally includes dipalmitoyl hydroxyproline, which makes up about 3% of the cream by weight. The cream additionally includes biosaccharide gum-1, which makes up about 5% of the cream by weight. The cream further includes one or more excipients including polyglyceryl-6 polyhydroxystearate as an emulsifier, where the one or more excipients make up the remainder of the cream by weight.

After the cream is applied to the vulvas of the 25 women, each of the 25 women reported improved moisture of their vulva, increased comfort in the skin of the vulva, a rejuvenated feeling of the skin of the vulva, an increased nourishment of the vulva, a lack of greasiness of the skin of the vulva, while 24 of the 25 women reported an improved health of the vulva and that the cream provided a protective barrier of the vulva. Additionally, 22 of the 25 women reported that the cream was absorbed quickly into the skin of the vulva, and 20 of the 25 women reported a pleasant scent of the vulva.

What is claimed is:

1. A composition, comprising:
an internal phase in an external phase,
wherein the internal phase, the external phase, or both comprise:
a prebiotic,
an antioxidant mixture, and
a moisturizer mixture, and
wherein the internal phase and the external phase are a reverse emulsion.

2. The composition of claim 1, wherein the composition is formulated for external administration to a skin region of a mammal.

3. The composition of claim 1 or 2, wherein the prebiotic comprises xylitol, lactitol, a fructan, a polysaccharide, an oligosaccharide, levan, polymnia sonchifolia root juice, or any combination of two or more thereof.

4. The composition of claim 3, wherein the fructan, the polysaccharide, and/or oligosaccharide comprise pectin, beta-glucan, inulin, bombax costatum flower extract, or any combination of two or more thereof.

5. The composition of any one of claims 1 to 4, wherein the antioxidant mixture comprises tetrahexyldecyl ascorbate, ubiquinone, resveratrol, epigallocatechin gallate, ellagic acid, ursolic acid, oleanolic acid, chlorogenic acid, rutin, hesperidin, quercetin, bakuchiol, botanical extracts of the leaves, flowers, seeds, fruits, stems, barks, or roots of a plant, vitamins, or any combination of two or more thereof.

6. The composition of claim 5, wherein,
the botanical extracts of the leaves, flowers, seeds, fruits, stems, barks, or roots of the plant comprise Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract, or any combination of two or more thereof, and
the vitamins or derivatives thereof comprise tocopherol, tocopheryl acetate, ubiquinone, ascorbic acid, magnesium ascorbyl phosphate, tetrahexyldecyl ascorbate, retinol, retinaldehyde, retinyl palmitate, or any combination of two or more thereof.

7. The composition of claim 6, wherein the antioxidant mixture comprises tocopherol.

8. The composition of any one of claims 1 to 7, wherein the moisturizer mixture comprises dimethicone, shea butter, jojoba esters, Helianthus annuus (sunflower) seed wax; Helianthus annuus (sunflower) seed oil, Simmondsia chinensis (jojoba) seed oil, Olea europaea (olive) oil, Glycine soja (soybean) oil, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, cetyl esters, or any combination of two or more thereof.

9. The composition of any one of claims 1 to 8, wherein the internal phase, external phase, or both comprises dipalmitoyl hydroxyproline.

10. The composition of any one of claims 1 to 9, wherein the internal phase, external phase, or both further comprise a carbohydrate or derivative thereof.

11. The composition of claim 10, wherein the carbohydrate comprises biosaccharide gum-1.

12. The composition of any one of claims 1 to 11, wherein the internal phase, external phase, or both further comprise one or more excipients.

13. The composition of claim 12, wherein the one or more excipients comprises caprylic/capric triglyceride, glycerin, squalane, dimethicone, glyceryl behenate/eicosadioate, magnesium sulfate, steareth-2, oleth-2, oleth-7, ceteareth-20, sorbitan oleate, sorbitan stearate, sorbitan isostearate, sorbitan laurate, sorbitan olivate, glyceryl oleate, glyceryl stearate, glyceryl ricinoleate, glyceryl caprylate, glyceryl undecylenate, sucrose distearate, peg-20 methyl glucose sesquistearate, peg-30 polyhydroxystearate, polyglyceryl-2 sesquiisostearate, polyglyceryl-2 sesquioleate, palmitic acid, polyglyceryl-2 sesquicaprylate, polyglyceryl-2 diisostearate, polyglyceryl-2 dipolyhydroxystearate, polyglyceryl-2 oleate, polyglyceryl-2 stearate, polyglyceryl-3 oleate, polyglyceryl-3 behenate, polyglyceryl-3 diisostearate, polyglyceryl-3 triolivate, polyglyceryl-3 ricinolate, diisostearoyl polyglyceryl-3 dimer dilinolate, polyglyceryl-3 polyricinoleate, polyglyceryl-3 dimer dilinoleate, polyglyceryl-3 sorbityl linseedate, polyglyceryl-3 polydimethylsiloxyethyl dimethicone, polyglyceryl-3 methylglucose dioleate, polyglyceryl-4 stearate, polyglyceryl-4 oleate, polyglyceryl-4 isostearate, polyglyceryl-4 diisostearate/polyhydroxy stearate/sebacate, polyglyceryl-4 olivate/polyricinolate, polyglyceryl-6 oleate, polyglyceryl-6 pentaoleate, olive oil polyglyceryl-6 esters, polyglyceryl-6 polyricinolate, polyglyceryl-6 polyhydroxystearate, polyglyceryl-10 hexaoleate, polyglyceryl-10 diolate, polyglyceryl-10 stearate, methyl glucose sesquistearate, methyl glucose dioleate hydrogenated lecithin, cetyl peg/ppg-10/1 dimethicone, peg-10 dimethicone, peg/ppg-20/15 dimethicone, peg/ppg-18/18 dimethicone, ppg-8 dimethicone, peg-9 methyl ether dimethicone, bis-peg/ppg-14/14 dimethicone, dimethicone/peg-10/15 crosspolymer, lauryl peg-9 polydimethylsiloxethyl dimethicone, lauryl dimethicone/polyglycerin-3 crosspolymer, lauryl peg-8 dimethicone, lauryl polyglyceryl-3 polydimethylsiloxethyl dimethicone, cetyl diglyceryl tris (trimethylsiloxy)silyethyl dimethicone, sodium hydroxide, ammonium hydroxide, triethanolamine, aminomethyl propanol, citric acid, phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, tetrasodium glutamate diacetate, disodium EDTA, tetrasodium edta, etidronic acid, trisodium dicarboxymethyl alaninate, trisodium ethylenediamine disuccinate, sodium phytate, or any combination of two or more thereof.

14. The composition of any one of claims 1 to 13, wherein the skin region is vulvar, penile, or scrotal.

15. The composition of any one of claims 2 to 14, wherein the mammal is a human.

16. The composition of claim 15, wherein the human is female.

17. The composition of any one of claims 14 to 16, wherein the skin region is vulvar.

18. The composition of claim 15, wherein the human is male.

19. The composition of any one of claim 14, 15, or 18, wherein the skin region is penile or scrotal.

20. The composition of any one of claims 1 to 19, wherein a level of moisture in the skin region is increased relative to a reference level of moisture of the skin region after externally administering the composition.

21. The composition of any one of claims 1 to 20, wherein a redness of the skin region is improved relative to a reference redness of the skin region after externally administering the composition.

22. The composition of any one of claims 1 to 21, wherein the composition is a formulation.

23. The composition of claim 22, wherein the formulation is a cream, lotion, serum, or solid-stick.

24. The composition of any one of claims 1 to 23, wherein the composition is formulated for topical administration to the skin region.

25. The composition of any one of claims 15 to 24, wherein the human experiencing menopausal symptoms, perimenopausal symptoms, premenopausal symptoms, or postmenopausal symptoms.

26. The composition of claim 25, wherein the menopausal symptoms, perimenopausal symptoms, premenopausal symptoms, or postmenopausal symptoms comprise dryness, irritation, itch, or redness of the skin region.

27. The composition of claim 25 or 26, wherein the menopausal symptoms, perimenopausal symptoms, premenopausal symptoms, or postmenopausal symptoms are reduced after administering the composition.

28. The composition of any one of claims 15 to 27, wherein the human is premenopausal.

29. The composition of any one of claims 1 to 28, wherein the composition is formulated to be administered at a dosage of about 0.01 grams to 10 grams.

30. The composition of any one of claims 1 to 29, wherein the prebiotic is present in the composition at about to about 0.001% to 5%

31. The composition of any one of claims 1 to 30, wherein the prebiotic is present in the composition at about 3% to 5%.

32. The composition of any one of claims 1 to 31, wherein the antioxidant mixture is present in the composition at about to about 0.001% to 5%.

33. The composition of any one of claims 1 to 32, wherein the antioxidant mixture is present in the composition at about 3% to 5%.

34. The composition of any one of claims 1 to 33, wherein the moisturizer mixture is present in the composition at about to about 0.001% to 80%.

35. The composition of any one of claims 1 to 34, wherein the moisturizer mixture is present in the composition at about to about 40% to 80%.

36. The composition of any one of claims 1 to 35, wherein the internal phase comprises a hydrator or humectant.

37. The composition of claim 36, wherein the hydrator or humectant comprises hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10, or any combination of two or more thereof.

38. The composition of any one of claims 1 to 37, wherein the internal phase is a water phase.

39. The composition of any one of claims 1 to 38, wherein the external phase is an oil phase or a silicone phase.

40. The composition of claims 1 to 39, wherein the internal phase, external phase, or both comprise the antioxidant mixture.

41. The composition of claim 40, wherein the antioxidant mixture comprises Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract, or any combination of two or more thereof.

42. The composition of any one of claims 1 to 41, wherein a level of smoothness of the skin region is improved relative to a reference level of smoothness of the skin region after externally administering the composition.

43. The composition of any one of claims 1 to 42, wherein a level of irritation of the skin region is improved relative to a reference level of irritation of the skin region after externally administering the composition.

44. The composition of any one of claims 1 to 43, wherein a texture of the skin region is improved relative to a reference texture of the skin region after externally administering the composition.

45. The composition of any one of claims 1 to 44, wherein the skin region is soothed after externally administering the composition.

46. The composition of any one of claims 1 to 45, wherein the composition is externally administered after the skin region is shaved.

47. The composition of any one of claims 1 to 46, wherein the internal phase comprises the antioxidant mixture.

48. The composition of any one of claims 1 to 46, wherein the external phase comprises the antioxidant mixture.

49. The composition of any one of claims 1 to 48, wherein the internal phase comprises the moisturizer mixture.

50. The composition of any one of claims 1 to 48, wherein the external phase comprises the moisturizer mixture.

51. The composition of any one of claims 1 to 50, wherein the internal phase comprises the carbohydrate.

52. The composition of any one of claims 1 to 50, wherein the external phase comprises the carbohydrate.

53. The composition of any one of claims 1 to 46, wherein:
a. the internal phase comprises the antioxidant mixture; and
b. the external phase comprises the moisturizer mixture.

54. The composition of claim 53, wherein the external phase comprises the carbohydrate.

55. A method for increasing moisture in a skin region of a mammal, comprising externally administering a composition formulated as a reverse emulsion to the skin region, the composition comprising:
an internal phase in an external phase, wherein the internal phase, external phase, or both comprise:
a prebiotic;
an antioxidant mixture; and
a moisturizer mixture.

56. The method of claim 55, wherein the prebiotic comprises xylitol, lactitol, a fructan, a polysaccharide, an oligosaccharide, levan, polymnia sonchifolia root juice, or any combination of two or more thereof.

57. The method of claim 56, wherein the fructan, the polysaccharide, and/or oligosaccharide comprise Pectin, Beta-Glucan, Inulin, Bombax Costatum Flower Extract, or any combination of two or more thereof.

58. The method of any one of claims 55 to 57, wherein the antioxidant mixture comprises tetrahexyldecyl ascorbate, ubiquinone, resveratrol, epigallocatechin gallate, ellagic acid, ursolic acid, oleanolic acid, chlorogenic acid, rutin, hesperidin, quercetin, bakuchiol, botanical extracts of the leaves, flowers, seeds, fruits, stems, barks, or roots of a plant, vitamins or derivatives thereof, or any combination of two or more thereof.

59. The method of claim 58, wherein,
the botanical extracts of the leaves, flowers, seeds, fruits, stems, barks, or roots of the plant comprise Camellia sinensis (green tea) extract, Vitis vinifera (grape) extract, Punica granatum (pomegranate) extract, Vaccinium vitis-idaea extract, Uterpe oleracea (açai) extract, Curcuma longa (turmeric) extract, Zingiber officinale (ginger) extract, Spinacia oleracea (spinach) extract, Olea europaea (olive) extract, or any combination of two or more thereof, and
the vitamins or derivatives thereof comprise tocopherol, tocoopheryl acetate, ubiquinone, ascorbic acid, magnesium ascorbyl phosphate, tetrahexyldecyl ascorbate, retinol, retinaldehyde, retinyl palmitate, or any combination of two or more thereof.

60. The method of any one of claims 55 to 59, wherein the antioxidant mixture comprises tocopherol.

61. The method of any one of claims 55 to 60, wherein the moisturizer mixture comprises dimethicone, shea butter, jojoba esters, Helianthus annuus (sunflower) seed wax; Helianthus annuus (sunflower) seed oil, Simmondsia chinensis (jojoba) seed oil, Olea europaea (olive) oil, Glycine soja (soybean) oil, Theobroma cacao (cocoa) seed butter, caprylic/capric triglyceride, squalane, phospholipids, dicaprylyl ether, octyldodecanol, ethylhexyl palmitate, isostearyl isostearate, octyldodecyl myristate, diisopropyl adipate, c12-15 alkyl benzoate, cetyl palmitate, decyl oleate, ethylhexyl cocoate, ethylhexyl stearate, isosorbide dicaprylate, cetyl esters, or any combination of two or more thereof.

62. The method of any one of claims 55 to 61, wherein the internal phase, external phase, or both further comprise dipalmitoyl hydroxyproline.

63. The method of any one of claims 55 to 62, wherein the internal phase, external phase, or both comprises a carbohydrate or derivative thereof.

64. The method of claim 63, wherein the carbohydrate comprises biosaccharide gum-1.

65. The method of any one of claims 55 to 64, wherein the internal phase, external phase, or both further comprise one or more excipients.

66. The method of claim 65, wherein the one or more excipients comprises caprylic/capric triglyceride, glycerin, squalane, dimethicone, glyceryl behenate/eicosadioate, magnesium sulfate, steareth-2, oleth-2, oleth-7, ceteareth-20, sorbitan oleate, sorbitan stearate, sorbitan isostearate, sorbitan laurate, sorbitan olivate, glyceryl oleate, glyceryl stearate, glyceryl ricinoleate, glyceryl caprylate, glyceryl undecylenate, sucrose distearate, peg-20 methyl glucose sesquistearate, peg-30 polyhydroxystearate, polyglyceryl-2 sesquiisostearate, polyglyceryl-2 sesquioleate, palmitic acid, polyglyceryl-2 sesquicaprylate, polyglyceryl-2 diisostearate, polyglyceryl-2 dipolyhydroxystearate, polyglyceryl-2 oleate, polyglyceryl-2 stearate, polyglyceryl-3 oleate, polyglyceryl-3 behenate, polyglyceryl-3 diisostearate, polyglyceryl-3 triolivate, polyglyceryl-3 ricinolate, diisostearoyl polyglyceryl-3 dimer dilinolate, polyglyceryl-3 polyricinoleate, polyglyceryl-3 dimer dilinoleate, polyglyceryl-3 sorbityl linseedate, polyglyceryl-3 polydimethylsiloxyethyl dimethicone, polyglyceryl-3 methylglucose dioleate, polyglyceryl-4 stearate, polyglyceryl-4 oleate, polyglyceryl-4 isostearate, polyglyceryl-4 diisostearate/polyhydroxy stearate/sebacate, polyglyceryl-4 olivate/polyricinolate, polyglyceryl-6 oleate, polyglyceryl-6 pentaoleate, olive oil polyglyceryl-6 esters, polyglyceryl-6 polyricinolate, polyglyceryl-6 polyhydroxystearate, polyglyceryl-10 hexaoleate, polyglyceryl-10 diolate, polyglyceryl-10 stearate, methyl glucose sesquistearate, methyl glucose dioleate hydrogenated lecithin, cetyl peg/ppg-10/1 dimethicone, peg-10 dimethicone, peg/ppg-20/15 dimethicone, peg/ppg-18/18 dimethicone, ppg-8 dimethicone, peg-9 methyl ether dimethicone, bis-peg/ppg-14/14 dimethicone, dimethicone/peg-10/15 crosspolymer, lauryl peg-9 polydimethylsiloxethyl dimethicone, lauryl dimethicone/polyglycerin-3 crosspolymer, lauryl peg-8 dimethicone, lauryl polyglyceryl-3 polydimethylsiloxethyl dimethicone, cetyl diglyceryl tris (trimethylsiloxy)silyethyl dimethicone, sodium hydroxide, ammonium hydroxide, triethanolamine, aminomethyl propanol, citric acid, phenoxyethanol, methylparaben, propylparaben, butylparaben, diazolidinyl urea, imidazolidinyl urea, iodopropynyl butylcarbamate, sodium hydroxymethylglycinate, sorbic acid and its derivatives, benzoic acid and its derivatives, ethylhexylglycerin, benzyl alcohol, caprylyl glycol, tetrasodium glutamate diacetate, disodium EDTA, tetrasodium edta, etidronic acid, trisodium dicarboxymethyl alaninate, trisodium ethylenediamine disuccinate, sodium phytate, or any combination of two or more thereof.

67. The method of any one of claims 55 to 66, wherein the skin region is vulvar, penile, or scrotal.

68. The method of any one of claims 56 to 67, wherein the mammal is a human.

69. The method of claim 68, wherein the human is female.

70. The method of any one of claims 67 to 69, wherein the skin region is vulvar.

71. The method of claim 68, wherein the human is male.

72. The method of any one of claim 67, 68, or 71, wherein the skin region is penile or scrotal.

73. The method of any one of claims 55 to 72, wherein administering the composition to the skin region comprises administering the composition to the skin region at a frequency of once daily, twice daily, thrice daily, four times daily, five times daily, or six times daily.

74. The method of claim 73, wherein the frequency is twice daily.

75. The method of any one of claims 55 to 74, wherein a level of moisture in the skin region is increased relative to a reference level of moisture in the skin region after externally administering the composition.

76. The method of any one of claims 55 to 75, wherein a redness of the skin region is improved relative to a reference redness of the skin region after externally administering the composition.

77. The method of any one of claims 55 to 76, wherein the composition is a formulation.

78. The method of claim 77, wherein the formulation is a cream, serum, lotion, or solid-stick.

79. The method of any one of claims 55 to 78, wherein externally administering the composition to the skin region comprises topically applying the composition to the skin region.

80. The method of any one of claims 68 to 79, wherein the human is experiencing menopausal symptoms, perimenopausal symptoms, premenopausal symptoms, or postmenopausal symptoms.

81. The method of claim 80, wherein the menopausal symptoms, perimenopausal symptoms, premenopausal symptoms, or postmenopausal symptoms comprise dryness, irritation, itch, or redness of the skin region.

82. The method of claim 55 or 81, wherein the menopausal symptoms, perimenopausal symptoms, premenopausal symptoms, or postmenopausal symptoms are reduced after administering the composition.

83. The method of claim 68, wherein the human is premenopausal.

84. The method of any one of claims 55 to 83, wherein externally administering the composition to the skin region comprises administering the composition at a dosage of about 0.1 grams to about 10 grams.

85. The method of any one of claims 55 to 84, wherein the prebiotic is present in the composition at about to about 0.001% to 5%

86. The method of any one of claims 55 to 85, wherein the prebiotic is present in the composition at about 3% to 5%.

87. The method of any one of claims 55 to 86, wherein the antioxidant mixture is present in the composition at about to about 0.001% to 5%.

88. The method of any one of claims 55 to 87, wherein the antioxidant mixture is present in the composition at about 3% to 5%.

89. The method of any one of claims 55 to 88, wherein the moisturizer mixture is present in the composition at about to about 0.001% to 80%.

90. The method of any one of claims 55 to 89, wherein the moisturizer mixture is present in the composition at about to about 40% to 80%.

91. The method of any one of claims 55 to 90, wherein externally administering the composition results in increased collagen production in the skin region.

92. The method of any one of claims 55 to 91, wherein the internal phase comprises a hydrator or humectant.

93. The method of claim 92, wherein the hydrator or humectant comprises hyaluronic acid and/or its derivatives, honey, aloe barbadensis leaf juice, urea, biosaccharide gum-1, saccharide isomerate, sodium pca, sodium lactate, polyols and/or their derivatives such as propylene glycol, butylene glycol, pentylene glycol, propanediol, isopentyldiol, 2,3-butanediol, 1,2-pentanediol, 1,2-heptanediol, glycerin, erythritol, xylitol, mannitol, sorbitol, PEG-4, PEG-8, PEG-32, methyl gluceth-10, methyl gluceth-20, glycereth-26 and/or polyglycerin-10, or any combination of two or more thereof.

94. The method of any one of claims 55 to 93, wherein the internal phase is a water phase.

95. The method of any one of claims 55 to 94, wherein the external phase is an oil phase or a silicone phase.

96. The method of claims 55 to 95, wherein the internal phase, external phase, or both comprises the antioxidant mixture.

97. The method of claim 96, wherein the antioxidant mixture comprises Bacillus ferment, Bacillus ferment extract, Bacillus ferment filtrate extract, Lactobacillus ferment, Lactobacillus ferment lysate, Saccharomyces lysate, Saccharomyces ferment lysate filtrate, Saccharomyces/zinc ferment, faex (yeast extract), Pseudoalteromonas ferment extract, Alteromonas ferment filtrate, Alteromonas ferment extract, or any combination of two or more thereof.

98. The method of any one of claims 55 to 97, wherein a level of smoothness of the skin region is improved relative to a reference level of smoothness of the skin region after externally administering the composition.

99. The method of any one of claims 55 to 98, wherein a level of irritation of the skin region is improved relative to a reference level of irritation of the skin region after externally administering the composition.

100. The method of any one of claims 55 to 99, wherein a texture of the skin region is improved relative to a reference texture of the skin region after externally administering the composition.

101. The method of any one of claims 55 to 100, wherein the skin region is soothed after externally administering the composition.

102. The method of any one of claims 55 to 101, wherein the composition is externally administered after the skin region is shaved.

103. The method of any one of claims 55 to 102, wherein the internal phase comprises the antioxidant mixture.

104. The method of any one of claims 55 to 102, wherein the external phase comprises the antioxidant mixture.

105. The method of any one of claims 55 to 104, wherein the internal phase comprises the moisturizer mixture.

106. The method of any one of claims 55 to 104, wherein the external phase comprises the moisturizer mixture.

107. The method of any one of claims 55 to 106, wherein the internal phase comprises the carbohydrate.

108. The method of any one of claims 55 to 106, wherein the external phase comprises the carbohydrate.

109. The method of any one of claims 55 to 108, wherein:
a. the internal phase comprises the antioxidant mixture; and
b. the external phase comprises the moisturizer mixture.

110. The method of claim 109, wherein the external phase comprises the carbohydrate.
